(19) United States
(12) Patent Application Publication
(10) Pub. No.: US 2010/0022589 A1
(43) Pub. Date: Jan. 28, 2010

(54) PYRIDINE-3-CARBOXAMIDE COMPOUNDS AND THEIR USE FOR INHIBITING 11-BETA-HYDROXYSTEROID DEHYDROGENASE

(76) Inventors: William McCoull, Cheshire (GB); Martin Packer, Cheshire (GB); James Stewart Scott, Cheshire (GB); Paul Robert Owen Whittamore, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 12/375,302

(22) PCT Filed: Jul. 26, 2007
(86) PCT No.: PCT/GB07/02831

§ 371 Date: Jan. 27, 2009

Related U.S. Application Data
(60) Provisional application No. 60/820,557, filed on Jul. 27, 2006.

(51) Int. Cl. A61K 31/4545; C07D 213/60; A61K 31/44; A61K 31/443; C07D 405/12; C07D 401/12; A61K 31/444; A61P 3/00

(52) U.S. Cl. 514/318; 546/298; 514/350; 514/336; 546/284.4; 546/261; 514/335; 546/194

(57) ABSTRACT

Compounds of formula (I):

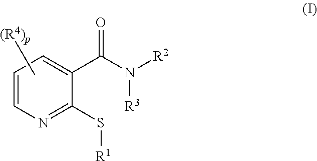

wherein variable groups are defined within; their use in the inhibition of 11βHSD1, processes for making them and pharmaceutical compositions comprising them are described.

This invention relates to chemical compounds, or pharmaceutically-acceptable salts thereof. These compounds possess human 11-β-hydroxysteroid dehydrogenase type 1 enzyme (11βHSD1) inhibitory activity and accordingly have value in the treatment of disease states including metabolic syndrome and are useful in methods of treatment of a warm-blooded animal, such as man. The invention also relates to processes for the manufacture of said compounds, to pharmaceutical compositions containing them and to their use in the manufacture of medicaments to inhibit 11βHSD1 in a warm-blooded animal, such as man.

Glucocorticoids (cortisol in man, corticosterone in rodents) are counter regulatory hormones i.e. they oppose the actions of insulin (Dallman M F, Strack A M, Akana S F et al. 1993; Front Neuroendocrinol 14, 303-347). They regulate the expression of hepatic enzymes involved in gluconeogenesis and increase substrate supply by releasing glycerol from adipose tissue (increased lipolysis) and amino acids from muscle (decreased protein synthesis and increased protein degradation). Glucocorticoids are also important in the differentiation of pre-adipocytes into mature adipocytes which are able to store triglycerides (Bujalska I J et al. 1999; Endocrinology 140, 3188-3196). This may be critical in disease states where glucocorticoids induced by “stress” are associated with central obesity which itself is a strong risk factor for type 2 diabetes, hypertension and cardiovascular disease (Bjorntorp P & Rosmond R 2000; Int. J. Obesity 24, S80-S85)

It is now well established that glucocorticoid activity is controlled not simply by secretion of cortisol but also at the tissue level by intracellular interconversion of active cortisol and inactive cortisone by the 11-beta hydroxysteroid dehydrogenases, 11βHSD1 (which activates cortisone) and 11βHSD2 (which inactivates cortisol) (Sandeep T C & Walker B R 2001 Trends in Endocrinol & Metab. 12, 446-453). That this mechanism may be important in man was initially shown using carbenoxolone (an anti-ulcer drug which inhibits both 11βHSD1 and 2) treatment which (Walker B R et al. 1995; J. Clin. Endocrinol. Metab. 80, 3155-3159) leads to increased insulin sensitivity indicating that 11βHSD1 may well be regulating the effects of insulin by decreasing tissue levels of active glucocorticoids (Walker B R et al. 1995; J. Clin. Endocrinol. Metab. 80, 3155-3159).

Clinically, Cushing's syndrome is associated with cortisol excess which in turn is associated with glucose intolerance, central obesity (caused by stimulation of pre-adipocyte differentiation in this depot), dyslipidaemia and hypertension. Cushing's syndrome shows a number of clear parallels with metabolic syndrome. Even though the metabolic syndrome is not generally associated with excess circulating cortisol levels (Jessop D S et al. 2001; J. Clin. Endocrinol. Metab. 86, 4109-4114) abnormally high 11βHSD1 activity within tissues would be expected to have the same effect. In obese men it was shown that despite having similar or lower plasma cortisol levels than lean controls, 11βHSD1 activity in subcutaneous fat was greatly enhanced (Rask E et al. 2001; J. Clin. Endocrinol. Metab. 1418-1421). Furthermore, the central fat, associated with the metabolic syndrome expresses much higher levels of 11βHSD1 activity than subcutaneous fat (Bujalska I J et al. 1997; Lancet 349, 1210-1213). Thus there appears to be a link between glucocorticoids, 11βHSD1 and the metabolic syndrome.

11βHSD1 knock-out mice show attenuated glucocorticoid-induced activation of gluconeogenic enzymes in response to fasting and lower plasma glucose levels in response to stress or obesity (Kotelevtsev Y et al. 1997; Proc. Natl. Acad. Sci. USA 94, 14924-14929) indicating the utility of inhibition of 11βHSD1 in lowering of plasma glucose and hepatic glucose output in type 2 diabetes. Furthermore, these mice express an anti-atherogenic lipoprotein profile, having low triglycerides, increased HDL cholesterol and increased apo-lipoprotein AI levels. (Morton N M et al. 2001; J. Biol. Chem. 276, 41293-41300). This phenotype is due to an increased hepatic expression of enzymes of fat catabolism and PPARα. Again this indicates the utility of 11βHSD1 inhibition in treatment of the dyslipidaemia of the metabolic syndrome.

The most convincing demonstration of a link between the metabolic syndrome and 11βHSD1 comes from recent studies of transgenic mice over-expressing 11βHSD1 (Masuzaki H et al. 2001; Science 294, 2166-2170). When expressed under the control of an adipose specific promoter, 11βHSD1 transgenic mice have high adipose levels of corticosterone, central obesity, insulin resistant diabetes, hyperlipidaemia and hyperphagia. Most importantly, the increased levels of 11βHSD1 activity in the fat of these mice are similar to those seen in obese subjects. Hepatic 11βHSD1 activity and plasma corticosterone levels were normal, however, hepatic portal vein levels of corticosterone were increased 3 fold and it is thought that this is the cause of the metabolic effects in liver.

Overall it is now clear that the complete metabolic syndrome can be mimicked in mice simply by overexpressing 11βHSD1 in fat alone at levels similar to those in obese man.

11βHSD1 tissue distribution is widespread and overlapping with that of the glucocorticoid receptor. Thus, 11βHSD1 inhibition could potentially oppose the effects of glucocorticoids in a number of physiological/pathological roles. 11βHSD1 is present in human skeletal muscle and glucocorticoid opposition to the anabolic effects of insulin on protein turnover and glucose metabolism are well documented (Whorwood C B et al. 2001; J. Clin. Endocrinol. Metab. 86, 2296-2308). Skeletal muscle must therefore be an important target for 11βHSD1 based therapy.

Glucocorticoids also decrease insulin secretion and this could exacerbate the effects of glucocorticoid induced insulin resistance. Pancreatic islets express 11βHSD1 and carbenoxolone can inhibit the effects of 11-dehydrocorticosterone on insulin release (Davani B et al. 2000; J. Biol. Chem. 275, 34841-34844). Thus in treatment of diabetes 11βHSD1 inhibitors may not only act at the tissue level on insulin resistance but also increase insulin secretion itself.

Skeletal development and bone function is also regulated by glucocorticoid action. 11βHSD1 is present in human bone osteoclasts and osteoblasts and treatment of healthy volunteers with carbenoxolone showed a decrease in bone resorption markers with no change in bone formation markers (Cooper M S et al 2000; Bone 27, 375-381). Inhibition of 11βHSD1 activity in bone could be used as a protective mechanism in treatment of osteoporosis.

Glucocorticoids may also be involved in diseases of the eye such as glaucoma. 11βHSD1 has been shown to affect intraocular pressure in man and inhibition of 11βHSD1 may be expected to alleviate the increased intraocular pressure associated with glaucoma (Rauz S et al. 2001; Investigative Opthalmology & Visual Science 42, 2037-2042).

There appears to be a convincing link between 11βHSD1 and the metabolic syndrome both in rodents and in humans. Evidence suggests that a drug which specifically inhibits 11βHSD1 in type 2 obese diabetic patients will lower blood glucose by reducing hepatic gluconeogenesis, reduce central obesity, improve the atherogenic lipoprotein phenotype, lower blood pressure and reduce insulin resistance. Insulin effects in muscle will be enhanced and insulin secretion from the beta cells of the islet may also be increased.

Currently there are two main recognised definitions of metabolic syndrome.

1) The Adult Treatment Panel (ATP III 2001 JMA) definition of metabolic syndrome indicates that it is present if the patient has three or more of the following symptoms:
Waist measuring at least 40 inches (102 cm) for men, 35 inches (88 cm) for women;
Serum triglyceride levels of at least 150 mg/dl (1.69 mmol/l);
HDL cholesterol levels of less than 40 mg/dl (1.04 mmol/l) in men, less than 50 mg/dl (1.29 mmol/l) in women;
Blood pressure of at least 135/80 mm Hg; and/or
Blood sugar (serum glucose) of at least 110 mg/dl (6.1 mmol/l).
2) The WHO consultation has recommended the following definition which does not imply causal relationships and is suggested as a working definition to be improved upon in due course:
The patient has at least one of the following conditions: glucose intolerance, impaired glucose tolerance (IGT) or diabetes mellitus and/or insulin resistance; together with two or more of the following:

### Raised Arterial Pressure;

Raised plasma triglycerides

### Central Obesity

### Microalbuminuria

We have found that the compounds defined in the present invention, or a pharmaceutically-acceptable salt thereof, are effective 11βHSD1 inhibitors, and accordingly have value in the treatment of disease states associated with metabolic syndrome. Accordingly there is provided a compound of formula (1):

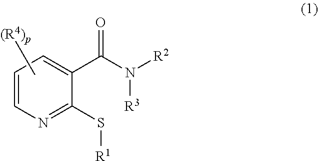

wherein:
R¹ is selected from phenylC₂₋₄alkyl, heteroarylC₂₋₄alkyl, phenylC₃₋₇cycloalkyl and heteroarylC₃₋₇cycloalkyl [each of which is optionally substituted on the ring, alkyl or cycloalkyl group by 1, 2 or 3 substitutents independently selected from C₁₋₃alkyl, C₂₋₃alkenyl, C₂₋₃alkynyl, hydroxy, halo, oxo, cyano, trifluoromethyl, C₁₋₃alkoxy, C₁₋₃alkylS(O)ₙ— (wherein n is 0, 1, 2 or 3), C₁₋₃alkylS(O)ₜO— (wherein t is 0, 1, 2 or 3), R⁵CON(R^{5″})—, (R^{5′})(R⁵″)NC(O)—, R^{5′}OC(O)— and (R^{5′})(R⁵″)NSO₂— (wherein R⁵ is C₁₋₃alkyl optionally substituted by hydroxyl, halo or cyano)]; and
R^{5′} and R^{5″} are independently selected from hydrogen and C₁₋₃alkyl optionally substituted by hydroxyl, halo, C₁₋₃alkoxy, carboxy or cyano)];
R² is selected from C₃₋₇cycloalkyl(CH₂)ₘ—, C₆₋₁₂bicycloalkyl(CH₂)ₘ— and C₆₋₁₂tricycloalkyl(CH₂)ₘ— (wherein m is 0, 1 or 2 and the cycloalkyl, bicycloalkyl and tricycloalkyl rings are optionally substituted by 1, 2 or 3 substituents independently selected from R⁶);
R³ is selected from hydrogen and C₁₋₄alkyl;
R² and R³ together with the nitrogen atom to which they are attached form a saturated mono, bicyclic or bridged ring system optionally containing 1 or 2 additional ring heteroatoms selected from nitrogen, oxygen and sulphur and which is optionally fused to a saturated, partially saturated or unsaturated monocyclic ring wherein the resulting ring system is optionally substituted by 1, 2, or 3 substituents independently selected from R⁷;
R⁴ is selected from C₁₋₄alkyl, C₂₋₄alkenyl, C₂₋₄alkynyl, hydroxyl, cyano, C₁₋₄alkanoyl, trifluoromethyl, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)_{q}—, C₁₋₄alkylS(O)_{q}C₁₋₄alkyl (wherein q is 0, 1 and 2) or a 4-7 membered saturated heterocyclic ring having 1 mandatory ring nitrogen and optionally an additional ring heteroatom selected from nitrogen, oxygen and sulphur (wherein any ring or alkyl group in each of the aforementioned groups is optionally substituted by 1, 2 or 3 substituents independently selected from R⁸);
R⁶, R⁷ and R⁸ are independently selected from hydroxyl, halo, oxo, carboxy, cyano, trifluoromethyl, R⁹, R⁹O—, R⁹CO—, R⁹C(O)O—, R⁹CON(R^{9′})—, (R^{9′})(R⁹″)NC(O)—, (R^{9′})(R⁹″)N—, R⁹S(O)ₐ— wherein a is 0 to 2, R^{9′}OC(O)—, (R^{9′})(R⁹″)NSO₂—, R⁹SO₂N(R^{9″})—, (R^{9′})(R^{9″})NC(O)N(R^{9′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted by 1, 2 or 3 substituents independently selected from C₁₋₄alkyl, hydroxyl, cyano, trifluoromethyl, trifluoromoxy, halo, C₁₋₄alkoxy, C₁₋₄alkoxyC₁₋₄alkyl, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ—, C₁₋₄alkylS(O)ᵣC₁₋₄alkyl (wherein r is 0, 1 and 2)];
R⁹ is C₁₋₃alkyl optionally substituted by hydroxyl, halo, C₁₋₄alkoxy, carboxy or cyano;
R^{9′}, R^{9″} and R^{9′″} are independently selected from hydrogen and C₁₋₃alkyl optionally substituted by hydroxyl, halo, C₁₋₄alkoxy, carboxy or cyano);
p is 0, 1 or 2;
or an in vivo hydrolysable ester or a pharmaceutically-acceptable salt thereof:
provided that the compound of the formula (1) is not:
1-{2-[(3,5-dimethyl-4-isoxazolyl)methylthio]-3-pyridylcarbonyl}-2-(2-thienyl)pyrrolidine;
N-cyclohexyl-2-(phenethylsulfanyl)-6-trifluoromethylpyridine-3-carboxamide; or
N-cyclohexyl-2-[2-(2-carboxyphenyl)ethyl)sulfanyl]pyridine-3-carboxamide.

In another aspect, the invention relates to a compound of the formula (1) as hereinabove defined or to a pharmaceutical salt thereof.

In another aspect there is provided a compound of formula (1) wherein:

R¹ is selected from phenylC₂₋₄alkyl, hetarylC₁₋₄alkyl, phenylC₃₋₇cycloalkyl and hetarylC₃₋₇cycloalkyl (each of which is optionally substituted by 1, 2 or 3 substituents independently selected from C₁₋₃alkyl, C₂₋₃alkenyl, C₂₋₃alkynyl, hydroxy, halo, oxo, cyano, trifluoromethyl, C₁₋₃alkoxy, C₁₋₃alkylS(O)ₙ— (wherein n is 0, 1, 2 or 3), R⁵CON(R^{5′})—, (R^{5′})(R⁵″)NC(O)—, R^{5′}OC(O)— and (R^{5′})(R⁵″)NSO₂— (wherein R⁵ is C₁₋₃alkyl optionally substituted by hydroxyl, halo or cyano; and
R^{5′} and R^{5″} are independently selected from hydrogen and C₁₋₃alkyl optionally substituted by hydroxyl, halo, C₁₋₄alkoxy, carboxy or cyano)] and
R² is selected from C₃₋₇cycloalkyl(CH₂)ₘ—, C₆₋₁₂bicycloalkyl(CH₂)ₘ— and C₆₋₁₂tricycloalkyl(CH₂)ₘ— (wherein m is 0, 1 or 2 and the cycloalkyl, bicycloalkyl and tricycloalkyl rings are optionally substituted by 1, 2 or 3 substituents independently selected from R⁶);
R³ is selected from hydrogen and C₁₋₄alkyl;
R² and R³ together with the nitrogen atom to which they are attached form a saturated mono, bicyclic or bridged ring system optionally containing 1 or 2 additional ring heteroatoms selected from nitrogen, oxygen and sulphur and which is optionally fused to a saturated, partially saturated or unsaturated monocyclic ring wherein the resulting ring system is optionally substituted by 1, 2, or 3 substituents independently selected from R⁷;
R⁴ is selected from C₁₋₄alkyl, C₂₋₄alkenyl, C₂₋₄alkynyl, hydroxyl, cyano, C₁₋₄alkanoyl, trifluoromethyl, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)_{q}—, C₁₋₄alkylS(O)_{q}C₁₋₄alkyl (wherein q is 0, 1 and 2) or a 4-7 membered saturated heterocyclic ring having 1 mandatory ring nitrogen and optionally an additional ring heteroatom selected from nitrogen, oxygen and sulphur (wherein each of the aforementioned groups is optionally substituted by 1, 2 or 3 substituents independently selected from R⁸);
R⁶, R⁷ and R⁸ are independently selected from hydroxyl, halo, oxo, carboxy, cyano, trifluoromethyl, R⁹, R⁹O—, R⁹CO—, R⁹C(O)O—, R⁹CON(R^{9′})—, (R^{9′})(R⁹″)NC(O)—, (R^{9′})(R⁹″)N—, R⁹S(O)ₐ— wherein a is 0 to 2, R^{9′}OC(O)—, (R^{9′})(R⁹″)NSO₂—, R⁹SO₂N(R^{9″})—, (R^{9′})(R^{9″})NC(O)N(R^{9′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted by 1, 2 or 3 substituents independently selected from C₁₋₄alkyl, hydroxyl, cyano, trifluoromethyl, trifluoromoxy, halo, C₁₋₄alkoxy, C₁₋₄alkoxyC₁₋₄alkyl, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ—, C₁₋₄alkylS(O)ᵣC₁₋₄alkyl (wherein r is 0, 1 and 2)];
R⁹ is C₁₋₃alkyl optionally substituted by hydroxyl, halo, C₁₋₄alkoxy, carboxy or cyano;
R^{9′}, R^{9″} and R^{9′″} are independently selected from hydrogen and C₁₋₃alkyl optionally substituted by hydroxyl, halo, C₁₋₄alkoxy, carboxy or cyano);
p is 0, 1 or 2;
or a pharmaceutically-acceptable salt thereof;
provided that the compound of the formula (1) is not:
N-cyclohexyl 2-(4-pyridylmethylthio)-3-pyridinecarboxamide;
4-[2-(4-pyridylmethylthio)-3-pyridylcarbonyl]morpholine; or
1-methyl-4-[2-(3-pyridylmethylthio)-3-pyridylcarbonyl]piperazine.

In another aspect, the invention relates to a compound of the formula (1) as hereinabove defined with the proviso that it is not 1-{2-[(3,5-dimethyl-4-isoxazolyl)methylthio]-3-pyridylcarbonyl}-2-(2-thienyl)pyrrolidine.

In this specification the term “alkyl” includes both straight and branched chain alkyl groups but references to individual alkyl groups such as “propyl” are specific for the straight chain version only. For example, “C₁₋₄alkyl” includes propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals therefore “C₁₋₄alkoxyC₁₋₄alkyl” would include 1-(C₁₋₄alkoxy)propyl, 2-(C₁₋₄alkoxy)ethyl and 3-(C₁₋₄alkoxy)butyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

“Heteroaryl”, unless otherwise specified, is a totally unsaturated, monocyclic ring containing 5 or 6 atoms of which at least 1, 2 or 3 ring atoms are independently chosen from nitrogen, sulphur or oxygen, which may, unless otherwise specified, be carbon or nitrogen linked. A ring nitrogen atom may be optionally oxidised to form the corresponding N-oxide. Examples and suitable values of the term “heteroaryl” are thienyl, furyl, thiazolyl, pyrazolyl, isoxazolyl, imidazolyl, pyrrolyl, thiadiazolyl, isothiazolyl, triazolyl, pyrimidyl, pyrazinyl, pyridazinyl and pyridyl. Particularly “heteroaryl” refers to thienyl, furyl, thiazolyl, pyridyl, imidazolyl or pyrazolyl.

A “saturated mono, bicyclic or bridged ring system optionally containing 1 or 2 additional Ting heteroatoms selected from nitrogen, oxygen and sulphur”, unless otherwise specified contains 4-14 ring atoms. Particularly a mono ring contains 4-7 ring atoms, a bicyclic ring 6-14 ring atoms and a bridged ring system 6-14 ring atoms. Examples of mono rings include piperidinyl, piperazinyl and morpholinyl. Examples of bicyclic rings include decalin and 2,3,3a,4,5,6,7,7a-octahydro-1H-indene.

Bridged ring systems are ring systems in which there are two or more bonds common to two or more constituent rings. Examples of bridged ring systems include 1,3,3-trimethyl-6-azabicyclo[3.2.1]octane, 2-aza-bicyclo[2.2.1]heptane and 7-azabicyclo[2,2,1]heptane, 1- and 2-adamantanyl.

A “saturated, partially saturated or unsaturated monocyclic ring” is, unless otherwise specified, a 4-7 membered ring. Examples include, cyclopropyl, cyclobutyl, cyclopentyl, cyclopentenyl, cyclohexyl, cyclohexenyl and phenyl.

Examples of the “4-7 membered saturated heterocyclic ring having 1 mandatory ring nitrogen and optionally an additional ring heteroatom selected from nitrogen, oxygen and sulphur” include piperidinyl, piperazinyl and morpholinyl.

Examples of a “saturated or partially-saturated 5- or 6-membered ring optionally containing 1, 2 or 3 heteroatoms independently selected from nitrogen, oxygen and sulphur” include piperidinyl, piperazinyl and morpholinyl.

Examples of “C₁₋₄alkoxy” include methoxy, ethoxy and propoxy. Examples of “C₁₋₄alkoxyC₁₋₄alkyl” include methoxymethyl, ethoxymethyl, propoxymethyl, 2-methoxyethyl, 2-ethoxyethyl and 2-propoxyethyl. Examples of “C₁₋₄alkylS(O)ₙ wherein n is 0 to 2” include methylthio, ethylthio, methylsulphinyl, ethylsulphinyl, mesyl and ethylsulphonyl. Examples of “C₁₋₄alkylS(O)_{q}C₁₋₄alkyl” wherein q is 0 to 2” include methylthio, ethylthio, methylsulphinyl, ethylsulphinyl, mesyl, ethylsulphonyl, methylthiomethyl, ethylthiomethyl, methylsulphinylmethyl, ethylsulphinylmethyl, mesylmethyl and ethylsulphonylmethyl. Examples of “C₁₋₄alkanoyl” include propionyl and acetyl. Examples of “N—(C₁₋₄alkyl)amino” include methylamino and ethylamino. Examples of “N,N—(C₁₋₄alkyl)₂-amino” include N,N-dimethylamino, N,N-diethylamino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl Examples of “N—(C₁₋₄alkyl)carbamoyl” are methylaminocarbonyl and ethylaminocarbonyl. Examples of “N,N—(C₁₋₄alkyl)₂-carbamoyl” are dimethylaminocarbonyl and methylethylaminocarbonyl. Examples of “C₃₋₇cycloalkyl(CH₂)ₘ—” include cyclopropymethyl, 2-cyclopropylethyl, cyclobutylmethyl, cyclopentylmethyl and cyclohexylmethyl. Examples of C₆₋₁₂bicycloalkyl(CH₂)ₘ— include norbornyl bicyclo[2.2.2]octane(CH₂)ₘ— and bicyclo[3.2.1]octane(CH₂)ₘ—. Examples of C₆₋₁₂tricycloalkyl(CH₂)ₘ— include 1- and 2-adamantanyl(CH₂)ₘ—.

An in-vivo hydrolysable ester of a compound of the invention containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁ to C₆alkoxymethyl esters for example methoxymethyl, C₁ to C₆alkanoyloxymethyl esters for example pivaloyloxymethyl, phthalidyl esters, C₃ to C₈cycloalkoxycarbonyloxyC₁ to C₆alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

An in-vivo hydrolysable ester of a compound of the invention containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

A suitable pharmaceutically-acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically-acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (1) may have chiral centres and/or geometric isomeric centres (E- and Z-isomers), and it is to be understood that the invention encompasses all such optical, diastereoisomers and geometric isomers that possess 11βHSD1 inhibitory activity.

The invention relates to any and all tautomeric forms of the compounds of the formula (1) that possess 11βHSD1 inhibitory activity.

It is also to be understood that certain compounds of the formula (1) can exist in solvated as well as unsolvated forms such as, for example, hydrated forms. It is to be understood that the invention encompasses all such solvated forms, which possess 11βHSD1 inhibitory activity.

In one embodiment of the invention are provided compounds of formula (1). In an alternative embodiment are provided pharmaceutically-acceptable salts of compounds of formula (1).

Particular values of variable groups are as follows. Such values may be used where appropriate with any of the definitions, claims or embodiments defined hereinbefore or hereinafter, for compounds of formula (1):

1. In another aspect, the invention relates to a compound of the formula (I) as hereinabove defined wherein R¹ is selected from phenylC₂₋₄alkyl, hetarylC₂₋₄alkyl, phenylC₃₋₇cycloalkyl and hetarylC₃₋₇cycloalkyl (each of which is optionally substituted by 1, 2 or 3 substituents independently selected from C₁₋₃alkyl, C₂₋₃alkenyl, C₂₋₃alkynyl, hydroxy, halo, oxo, cyano, trifluoromethyl, C₁₋₃alkoxy, C₁₋₃alkylS(O)ₙ— (wherein n is 0, 1, 2 or 3), C₁₋₃alkylS(O)ₜO— (wherein t is 0, 1, 2 or 3), R⁵CON(R^{5′})—, (R^{5′})(R⁵″)NC(O)—, R^{5′}OC(O)— and (R^{5′})(R⁵″)NSO₂— (wherein R⁵ is C₁₋₃alkyl optionally substituted by hydroxyl, halo or cyano; and
R^{5′} and R^{5″} are independently selected from hydrogen and C₁₋₃alkyl optionally substituted by hydroxyl, halo, C₁₋₄alkoxy, carboxy or cyano)].
2. In yet another aspect, the invention relates to a compound of the formula (I) as hereinabove defined wherein R¹ is selected from phenylC₂₋₃alkyl and hetarylC₂₋₃alkyl, (each of which is optionally substituted by 1, 2 or 3 substituents independently selected from C₁₋₃alkyl, C₂₋₃alkenyl, C₂₋₃alkynyl, hydroxy, halo, oxo, cyano, trifluoromethyl, C₁₋₃alkoxy, C₁₋₃alkylS(O)ₙ— (wherein n is 0, 1, 2 or 3), C₁₋₃alkylS(O)ₜO— (wherein t is 0, 1, 2 or 3), R⁵CON(R^{5′})—, (R^{5′})(R⁵″)NC(O)—, R^{5′}OC(O)— and (R^{5′})(R⁵″)NSO₂— (wherein R⁵ is C₁₋₃alkyl optionally substituted by hydroxyl, halo or cyano; and
R^{5′} and R^{5″} are independently selected from hydrogen and C₁₋₃alkyl optionally substituted by hydroxyl, halo, C₁₋₄alkoxy, carboxy or cyano)].
3. In yet another aspect, the invention relates to a compound of the formula (I) as hereinabove defined wherein R¹ is phenylethyl or 2-(pyridyl)ethyl (each being optionally substituted by 1, 2 or 3 substituents independently selected from C₁₋₃alkyl, C₂₋₃alkenyl, C₂₋₃alkynyl, hydroxy, halo, oxo, cyano, trifluoromethyl, C₁₋₃alkoxy, C₁₋₃alkylS(O)ₙ— (wherein n is 0, 1, 2 or 3), C₁₋₃alkylS(O)ₜO— (wherein t is 0, 1, 2 or 3), R⁵CON(R^{5′})—, (R^{5′})(R⁵″)NC(O)—, R^{5′}OC(O)— and (R^{5′})(R⁵″)NSO₂— (wherein R⁵ is C₁₋₃alkyl optionally substituted by hydroxyl, halo or cyano; and
R^{5′} and R^{5″} are independently selected from hydrogen and C₁₋₃alkyl optionally substituted by hydroxyl, halo, C₁₋₄alkoxy, carboxy or cyano)].
4. In one aspect, substituents from R¹ are selected from hydroxy, C₁₋₃alkoxy, C₁₋₃alkylS(O)ₙ— (wherein n is 0, 1, 2 or 3), C₁₋₃alkylS(O)ₜO— (wherein t is 0, 1, 2 or 3), and R^{5′}OC(O)— (wherein R⁵ is C₁₋₃alkyl optionally substituted by hydroxyl, halo or cyano; and R^{5′} and R^{5″} are independently selected from hydrogen and C₁₋₃alkyl optionally substituted by hydroxyl, halo, C₁₋₄alkoxy, carboxy or cyano).
5. In another aspect, substituents from R¹ are selected from R^{5′}OC(O)— (wherein R⁵ is C₁₋₃alkyl optionally substituted by hydroxyl, halo or cyano; and R^{5′} and R^{5″} are independently selected from hydrogen and C₁₋₃alkyl optionally substituted by hydroxyl, halo, C₁₋₄alkoxy, carboxy or cyano).
6. In one aspect the phenylC₂₋₄alkyl, hetarylC₁₋₄alkyl, phenylC₃₋₇cycloalkyl and hetarylC₃₋₇cycloalkyl groups in R¹ are optionally substituted by 1 or 2 substituents.
7. In one aspect, R³ is C₁₋₄alkyl.
8. In another aspect, R³ is hydrogen, methyl or ethyl.
9. In another aspect, R³ is hydrogen.
10. In another aspect, R³ is methyl.
11. In another aspect, R³ is ethyl.
12. In yet another aspect, the invention relates to a compound of the formula (I) as hereinabove defined with the proviso that R⁴ is in the 5-position of the pyridine ring it is not chloro.
13. In one aspect, when R⁴ is in the 5-position of the pyridine ring, it is hydrogen fluoro.
14. In one aspect, R⁴ is not in the 4-position of the pyridine ring.
15. In another aspect, R⁴ is not in the 5-position of the pyridine ring.
16. In one aspect, R⁴ is in the 6-position of the pyridine ring.
17. In one aspect, when R⁴ is in the 6-position of the pyridine ring it is amino, N—C₁₋₄alkylamino or di-N,N—(C₁₋₄alkyl)amino.
18. In one aspect, when R⁴ is in the 6-position of the pyridine ring it is methyl.
19. In one aspect, when R⁴ is in the 6-position of the pyridine ring it is chloro.
20. In one aspect, p is 1 or 2.
21. In another aspect, p is 0 or 1.
22. In another aspect, p is 1.
23. In another aspect, p is 2.
24. In one aspect, R² is selected from C₅₋₇cycloalkyl(CH₂)ₘ—, C₇₋₁₀bicycloalkyl(CH₂)ₘ— and C₈₋₁₂tricycloalkyl(CH₂)ₘ— (wherein the cycloalkyl, bicycloalkyl and tricycloalkyl rings are optionally substituted by 1, 2 or 3 substituents independently selected from R⁶) and wherein m is 0, 1 or 2.
25. In another aspect, R² is selected from C₅₋₇cycloalkyl(CH₂)ₘ—, C₇₋₁₀bicycloalkyl(CH₂)ₘ— and C₁₀tricycloalkyl(CH₂)ₘ— (wherein the cycloalkyl, bicycloalkyl and tricycloalkyl rings are optionally substituted by 1, 2 or 3 substituents independently selected from R⁶) and wherein m is 0, 1 or 2.
26. In yet another aspect, R² is selected from C₅₋₇cycloalkyl(CH₂)ₘ—, C₇₋₁₀bicycloalkyl(CH₂)ₘ— and adamantyl (wherein the cycloalkyl, bicycloalkyl and tricycloalkyl rings are optionally substituted by 1, 2 or 3 substituents independently selected from R⁶) and wherein m is 0, 1 or 2.
27. In yet another aspect, R² is adamantly (optionally substituted by 1, 2 or 3 substituents independently selected from R⁶).
28. In yet another aspect, R² is adamantyl (optionally substituted by 1 substituent selected from hydroxy and carboxy).
29. In yet another aspect, R² is cyclohexyl (optionally substituted by 1, 2 or 3 substituents independently selected from R⁶).
30. In yet another aspect, R² is cyclohexyl (optionally substituted by 1 carboxy group).
31. In one aspect, m is 0 or 1.
32. In another aspect, R² and R³ together with the nitrogen atom to which they are attached form a saturated 5 or 6-membered mono, 6-12 membered bicyclic or 6-12 membered bridged ring system optionally containing 1 or 2 additional ring heteroatoms selected from nitrogen, oxygen and sulphur and which is optionally fused to a saturated, partially-saturated or aryl monocyclic ring wherein the resulting ring system is optionally substituted by 1, 2, or 3 substituents independently selected from R⁷.
33. In one aspect, R⁶ is independently selected from hydroxyl, R⁹O—, R⁹CO— and R⁹C(O)O— wherein R⁹ is as hereinabove defined.
34. In one aspect, R⁶ is independently selected from hydroxyl, R⁹O—, R⁹CO— and R⁹C(O)O— wherein R⁹ is C₁₋₃alkyl optionally substituted by C₁₋₄alkoxy or carboxy.
35. In another aspect, R⁶ is independently selected from R⁹CON(R^{9′})—, R⁹SO₂N(R^{9″})— and (R^{9′})(R^{9″})NC(O)N(R^{9′″})—;
wherein R⁹ is as hereinabove defined.
36. In another aspect, R⁶ is independently selected from R⁹CON(R^{9′})—, R⁹SO₂N(R^{9″})— and (R^{9′})(R^{9″})NC(O)N(R^{9′″})—;
R⁹ is C₁₋₃alkyl optionally substituted by C₁₋₄alkoxy or carboxy;
R^{9′}, R^{9″} and R^{9′″} are independently selected from hydrogen and C₁₋₃alkyl optionally substituted by C₁₋₄alkoxy or carboxy).
37. In another aspect, R⁶ is independently selected from (R^{9′})(R⁹″)NC(O)— and (R^{9′})(R⁹″)N—; wherein R^{9′} and R^{9″} are as hereinabove defined.
38. In another aspect, R⁶ is independently selected from (R^{9′})(R⁹″)NC(O)— and (R^{9′})(R⁹″)N—; wherein R^{9′} and R^{9″} are independently selected from hydrogen and C₁₋₃alkyl optionally substituted by C₁₋₄alkoxy or carboxy.
39. In one aspect R⁶ is selected from hydroxy, carboxy, methyl, trifluoromethyl, chloro, fluoro, bromo, methoxy, ethoxy, trifluormethoxy, methanesulfonyl, ethanesulfonyl, methylthio, ethylthio, amino, N-methylamino, N-ethylamino, N-propylamino, N,N-dimethylamino, N,N-methylethylamino or N,N-diethylamino.
40. In another aspect, R⁶ is phenyl, pyridyl or pyrimidyl.
41. In another aspect, R⁶ is pyrid-2-yl, pyrid-3-yl or pyrid-4-yl.
42. In another aspect, R⁶ is hydroxy or carboxy.
43. In one aspect, R⁷ is independently selected from hydroxyl, R⁹, R⁹O—, R⁹CO— and R⁹C(O)O—
wherein R⁹ is as hereinabove defined.
44. In one aspect, R⁷ is independently selected from hydroxyl, R⁹, R⁹O—, R⁹CO— and R⁹C(O)O—
wherein R⁹ is C₁₋₃alkyl optionally substituted by C₁₋₄alkoxy or carboxy.
45. In another aspect, R⁷ is independently selected from R⁹CON(R^{9′})—, R⁹SO₂N(R^{9″})— and (R^{9′})(R^{9″})NC(O)N(R^{9′″})—;
wherein R⁹ is as hereinabove defined.
46. In another aspect, R⁷ is independently selected from R⁹CON(R^{9′})—, R⁹SO₂N(R^{9″})— and (R^{9′})(R^{9″})NC(O)N(R^{9′″})—;
R⁹ is C₁₋₃alkyl optionally substituted by C₁₋₄alkoxy or carboxy;
R^{9′}, R^{9″} and R^{9′″} are independently selected from hydrogen and C₁₋₃alkyl optionally substituted by C₁₋₄alkoxy or carboxy).
47. In another aspect, R⁷ is independently selected from (R^{9′})(R⁹″)NC(O)— and (R^{9′})(R⁹″)N—; wherein R^{9′} and R^{9″} are as hereinabove defined.
48. In another aspect, R⁷ is independently selected from (R^{9′})(R⁹″)NC(O)— and (R^{9′})(R⁹″)N—; wherein R^{9′} and R^{9″} are independently selected from hydrogen and C₁₋₃alkyl optionally substituted by C₁₋₄alkoxy or carboxy.
49. In one aspect R⁷ is selected from methyl, trifluoromethyl, chloro, fluoro, bromo, methoxy, ethoxy, hydroxyethyl, trifluormethoxy, methanesulfonyl, ethanesulfonyl, methylthio, ethylthio, amino, N-methylamino, N-ethylamino, N-propylamino, N,N-dimethylamino, N,N-methylethylamino or N,N-diethylamino.
50. In one aspect, R⁸ is independently selected from hydroxyl, R⁹O—, R⁹CO— and R⁹C(O)O— wherein R⁹ is as hereinabove defined.
51. In one aspect, R⁸ is independently selected from hydroxyl, R⁹O—, R⁹CO— and R⁹C(O)O— wherein R⁹ is C₁₋₃alkyl optionally substituted by C₁₋₄alkoxy or carboxy.
52. In another aspect, R⁸ is independently selected from R⁹CON(R^{9′})—, R⁹SO₂N(R^{9″})— and (R^{9′})(R^{9″})NC(O)N(R^{9′″})—;
wherein R⁹ is as hereinabove defined.
53. In another aspect, R⁸ is independently selected from R⁹CON(R^{9′})—, R⁹SO₂N(R^{9″})— and (R^{9′})(R^{9″})NC(O)N(R^{9′″})—;
R⁹ is C₁₋₃alkyl optionally substituted by C₁₋₄alkoxy or carboxy;
R^{9′}, R^{9″} and R^{9′″} are independently selected from hydrogen and C₁₋₃alkyl optionally substituted by C₁₋₄alkoxy or carboxy).
54. In another aspect, R⁸ is independently selected from (R^{9′})(R⁹″)NC(O)— and (R^{9′})(R⁹″)N—; wherein R^{9′} and R^{9″} are as hereinabove defined.
55. In another aspect, R⁸ is independently selected from (R^{9′})(R⁹″)NC(O)— and (R^{9′})(R⁹″)N—; wherein R^{9′} and R^{9″} are independently selected from hydrogen and C₁₋₃alkyl optionally substituted by C₁₋₄alkoxy or carboxy.
56. In one aspect R⁸ is selected from methyl, trifluoromethyl, chloro, fluoro, bromo, methoxy, ethoxy, trifluormethoxy, methanesulfonyl, ethanesulfonyl, methylthio, ethylthio, amino, N-methylamino, N-ethylamino, N-propylamino, N,N-dimethylamino, N,N-methylethylamino or N,N-diethylamino.
57. In one aspect R⁴ is selected from C₁₋₄alkyl, hydroxyl, cyano, C₁₋₄alkanoyl, trifluoromethyl, halo, C₁₋₄alkoxy, C₁₋₄alkoxyC₁₋₄alkyl, N—C₁₋₄alkylamino, di-N,N—(C₁₋₄alkyl)amino or a 4-7 membered saturated heterocyclic ring having 1 mandatory ring nitrogen and optionally an additional ring heteroatom selected from nitrogen, oxygen and sulphur (wherein any ring or alkyl group in each of the aforementioned groups is optionally substituted by 1, 2 or 3 substituents independently selected from R⁸).
58. In another aspect R⁴ is selected from C₁₋₄alkyl, trifluoromethyl, halo, C₁₋₄alkoxy, C₁₋₄alkoxyC₁₋₄alkyl, N—C₁₋₄alkylamino, di-N,N—(C₁₋₄alkyl)amino or a 4-7 membered saturated heterocyclic ring having 1 mandatory ring nitrogen and optionally an additional ring heteroatom selected from nitrogen, oxygen and sulphur (wherein any ring or alkyl group in each of the aforementioned groups is optionally substituted by 1, 2 or 3 substituents independently selected from R⁸).
59. In yet another aspect R⁴ is selected from C₁₋₄alkyl, trifluoromethyl and halo.
60. In one aspect, R⁴ is selected from methyl, fluoro and chloro.
61. In one aspect the compound of the formula (1) contains at least one carboxy group.

Further particular classes of compounds of the present invention are those of the formula (1) wherein R¹, R², R³, R⁴, R⁶, R⁷, R⁸ and p are as hereinabove defined in Table A using combinations of the definitions described hereinabove. For example, ‘1’ in the column headed R¹ in the table refers to definition 1. given for R¹ hereinabove and ‘I’ refers to the first definition given for the variables in the compound of formula (I) at the beginning of the description.

TABLE A

| | | | | R² | | | | | |
|---|---|---|---|---|---|---|---|---|---|
| | | | | and | | | | | |
| Class | R¹ | R² | R³ | R³ | R⁴ | p | R⁶ | R⁷ | R⁸ |
| 1 | 1 | 22 | 8 | — | I | I | I | — | I |
| 2 | 1 | — | — | 26 | I | I | — | I | I |
| 3 | 2 | 22 | 8 | — | 16 | I | I | — | 56 |
| 4 | 2 | — | — | 26 | 57 | 19 | — | 49 | 56 |
| 5 | 2 | 23 | 8 | — | 58 | 19 | 33 | — | 56 |
| | | | | | and | | | | |
| | | | | | 16 | | | | |
| 6 | 3 | 24 | 9 | — | 59 | 10 | 33 | — | — |
| | | | | | and | | | | |
| | | | | | 17 | | | | |

In another aspect, there is provided a compound of formula (1′):

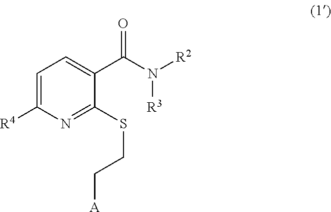

wherein:
A is selected from phenyl or heteroaryl [each of which is optionally substituted by 1, 2 or 3 substitutents independently selected from C₁₋₃alkyl, C₂₋₃alkenyl, C₂₋₃alkynyl, hydroxy, halo, oxo, cyano, trifluoromethyl, C₁₋₃alkoxy, C₁₋₃alkylS(O)ₙ— (wherein n is 0, 1, 2 or 3), C₁₋₃alkylS(O)ₜO— (wherein t is 0, 1, 2 or 3), R⁵CON(R^{5′})—, (R^{5′})(R⁵″)NC(O)—, R^{5′}OC(O)— and (R^{5′})(R⁵″)NSO₂— (wherein R⁵ is C₁₋₃alkyl optionally substituted by hydroxyl, halo or cyano)]; and
R^{5′} and R^{5″} are independently selected from hydrogen and C₁₋₃alkyl optionally substituted by hydroxyl, halo, C₁₋₃alkoxy, carboxy or cyano)];
R² is selected from C₃₋₇cycloalkyl(CH₂)ₘ—, C₆₋₁₂bicycloalkyl(CH₂)ₘ— and C₆₋₁₂tricycloalkyl(CH₂)ₘ— (wherein m is 0, 1 or 2 and the cycloalkyl, bicycloalkyl and tricycloalkyl rings are optionally substituted by 1, 2 or 3 substituents independently selected from R⁶);
R³ is selected from hydrogen and methyl; or
R² and R³ together with the nitrogen atom to which they are attached form a saturated mono, bicyclic or bridged ring system optionally containing 1 or 2 additional ring heteroatoms selected from nitrogen, oxygen and sulphur and which is optionally fused to a saturated, partially saturated or unsaturated monocyclic ring wherein the resulting ring system is optionally substituted by 1, 2, or 3 substituents independently selected from R⁷;
R⁴ is selected from C₁₋₄alkyl, C₂₋₄alkenyl, C₂₋₄alkynyl, hydroxyl, cyano, C₁₋₄alkanoyl, trifluoromethyl, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)_{q}—, C₁₋₄alkylS(O)_{q}C₁₋₄alkyl (wherein q is 0, 1 and 2) or a 4-7 membered saturated heterocyclic ring having 1 mandatory ring nitrogen and optionally an additional ring heteroatom selected from nitrogen, oxygen and sulphur (wherein any ring or alkyl group in each of the aforementioned groups is optionally substituted by 1, 2 or 3 substituents independently selected from R⁸);
R⁶, R⁷ and R⁸ are independently selected from hydroxyl, halo, oxo, carboxy, cyano, trifluoromethyl, R⁹, R⁹O—, R⁹CO—, R⁹C(O)O—, R⁹CON(R^{9′})—, (R^{9′})(R⁹″)NC(O)—, (R^{9′})(R⁹″)N—, R⁹S(O)ₐ— wherein a is 0 to 2, R^{9′}OC(O)—, (R^{9′})(R⁹″)NSO₂—, R⁹SO₂N(R^{9″})—, (R^{9′})(R^{9″})NC(O)N(R^{9′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted by 1, 2 or 3 substituents independently selected from C₁₋₄alkyl, hydroxyl, cyano, trifluoromethyl, trifluoromoxy, halo, C₁₋₄alkoxy, C₁₋₄alkoxyC₁₋₄alkyl, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ—, C₁₋₄alkylS(O)ᵣC₁₋₄alkyl (wherein r is 0, 1 and 2)];
R⁹ is C₁₋₃alkyl optionally substituted by hydroxyl, halo, C₁₋₄alkoxy, carboxy or cyano;
R^{9′}, R^{9″} and R^{9′″} are independently selected from hydrogen and C₁₋₃alkyl optionally substituted by hydroxyl, halo, C₁₋₄alkoxy, carboxy or cyano);
or an in vivo hydrolysable ester or a pharmaceutically-acceptable salt thereof:
provided that the compound of the formula (1) is not:
N-cyclohexyl-2-(phenethylsulfanyl)-6-trifluoromethylpyridine-3-carboxamide; or
N-cyclohexyl-2-[2-(2-carboxyphenyl)ethyl)sulfanyl]pyridine-3-carboxamide;
   or an in vivo hydrolysable ester or a pharmaceutically-acceptable salt thereof.

In another aspect of the invention, suitable compounds of the invention are any one or more of the following compounds:
N-cyclohexyl-2-(3-phenylpropylsulfanyl)pyridine-3-carboxamide;
N-cyclohexyl-2-(2-furylmethylsulfanyl)pyridine-3-carboxamide;
N-cyclohexyl-2-(2-pyridin-2-ylethylsulfanyl)pyridine-3-carboxamide;
N-cyclohexyl-2-phenethylsulfanyl-pyridine-3-carboxamide;
N-cyclohexyl-2-(2-phenylpropylsulfanyl)pyridine-3-carboxamide N-Cyclohexyl-5-fluoro-2-phenethylsulfanyl-pyridine-3-carboxamide;
N-cyclohexyl-5-fluoro-2-phenethylsulfanyl-pyridine-3-carboxamide;
N-cyclohexyl-2-phenacylsulfanyl-pyridine-3-carboxamide;
N-cyclohexyl-2-(2-hydroxy-2-phenyl-ethyl)sulfanyl-pyridine-3-carboxamide;
N-cyclohexyl-2-[2-(4-methylsulfonyloxyphenyl)ethylsulfanyl]pyridine-3-carboxamide;
[3-(2-hydroxyethyl)-1-piperidyl]-(2-phenethylsulfanylpyridin-3-yl)methanone;
N-cyclohexyl-2-(2-pyridin-3-ylethylsulfanyl)pyridine-3-carboxamide;
6-chloro-N-cyclohexyl-2-phenethylsulfanyl-pyridine-3-carboxamide;
N-cyclohexyl-2-[2-(2-methylsulfonyloxyphenyl)ethylsulfanyl]pyridine-3-carboxamide;
N-cyclohexyl-2-[2-(2-hydroxyphenyl)ethylsulfanyl]pyridine-3-carboxamide;
2-[2-(3-carbamoylphenyl)ethylsulfanyl]-N-cyclohexyl-pyridine-3-carboxamide;
3-[2-[3-(cyclohexylcarbamoyl)pyridin-2-yl]sulfanylethyl]benzoic acid;
cis-4-[(6-methyl-2-phenethylsulfanyl-pyridine-3-carbonyl)amino]cyclohexane-1-carboxylic acid;
4-[methyl-(6-methyl-2-phenethylsulfanyl-pyridine-3-carbonyl)amino]cyclohexane-1-carboxylic acid;
6-chloro-N-[(2r,5s)-5-hydroxy-2-adamantyl]-2-phenethylsulfanyl-pyridine-3-carboxamide; and
(1r,4s)-4-[({6-methyl-2-[(2-phenylethyl)thio]pyridin-3-yl}carbonyl)amino]adamantane-1-carboxylic acid;
   or a pharmaceutically-acceptable salt thereof.

Another aspect of the present invention provides a process for preparing a compound of formula (1) or a pharmaceutically acceptable salt thereof which process [wherein variable groups are, unless otherwise specified, as defined in formula (1)] comprises any one of processes a) to c):

a) reaction of a compound of Formula (2) with a compound of Formula (3):

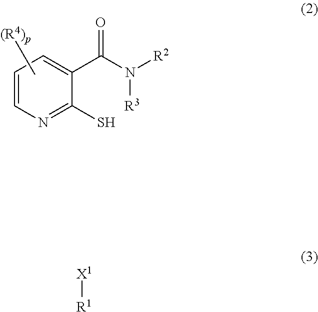

wherein X¹ is a leaving group; or
b) reaction of a compound of Formula (4) with a compound of Formula (5):

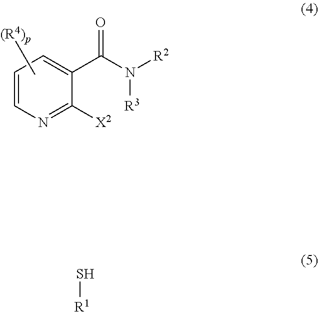

wherein X² is a leaving group; or
c) reaction of a compound of Formula (6) with a compound of Formula (7):

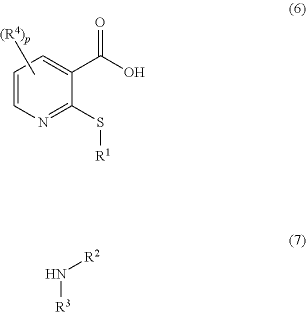

and thereafter if necessary or desirable:
i) converting a compound of the formula (1) into another compound of the formula (1);
ii) removing any protecting groups;
iii) resolving enantiomers;
iv) forming a salt or in vivo hydrolysable thereof.

Examples of conversions of a compound of Formula (1) into another compound of Formula (1), well known to those skilled in the art, include functional group interconversions such as hydrolysis, hydrogenation, hydrogenolysis, oxidation or reduction, and/or further functionalisation by standard reactions such as amide or metal-catalysed coupling, or nucleophilic displacement reactions.

Suitable conditions for the above processes a) to c) are as follows.

Process a) may be carried out in a suitable solvent such as acetonitrile, butyronitrile or methanol for example, typically with the addition of a suitable base such as potassium carbonate or sodium hydroxide for example. Typically the reaction is carried out at elevated temperature, using Microwave or conventional heating, for example at temperatures between 100-140° C. In certain cases the reactions can be carried out at ambient temperature. Suitable examples of leaving groups for process a) (X¹) are chloro, bromo, iodo, mesylate, tosylate or triflate. Others are known to the art.

Compounds of formula (2) may be made by processes known in the art and typically by reaction of a compound of Formula (8) with a compound of Formula (7):

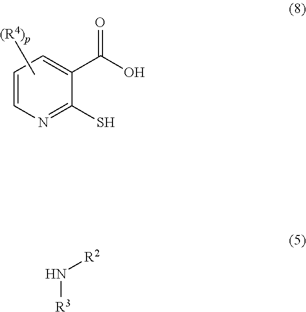

Such reactions may be carried out in a suitable solvent such as dichloromethane for example with the addition of a suitable coupling agent (or combination of agents) such as HOBT and EDCI for example, optionally in the presence of a suitable base such as triethylamine or N,N-di-iso-propylamine for example. Typically the reaction is carried out at ambient or elevated temperature between 0-60° C.

Process b) may be carried out in a suitable solvent such as acetonitrile, butyronitrile for example, typically with the addition of a suitable base such as potassium carbonate for example. Typically the reaction is carried out at ambient or elevated temperature between 0-60° C. Suitable examples of leaving groups for process b) are fluoro, and chloro. Others are known to the art.

Compounds of formula (4) may be made by processes known in the art and typically by reaction of a compound of Formula (9) with a compound of Formula (7):

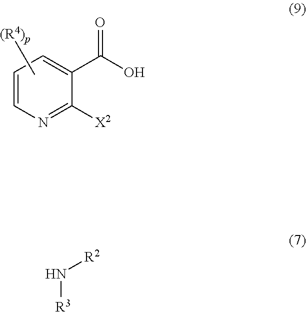

wherein the X² is hereinabove defined.

Such reactions may be carried out in a suitable solvent such as dichloromethane for example with the in situ formation of the acyl halide using a suitable reagent such as oxalyl chloride for example. Amide formation is carried out in the presence of a suitable base such as triethylamine or N,N-di-iso-propylamine for example. Typically the reaction is carried out at ambient or elevated temperature between 0-60° C.

Process c) is typically carried out in a suitable solvent such as dichloromethane for example with either the in situ formation of the acyl halide using a suitable reagent such as oxalyl chloride for example or with the addition of a suitable coupling agent (or combination of agents) to form an active ester such as HOBT and EDAC for example, optionally in the presence of a suitable base such as triethylamine or N,N-di-iso-propylamine for example. Typically the reaction is carried out at ambient or elevated temperature between 0-60° C.

Compounds of formula (6) may be made by processes known in the art and typically by reaction of a compound of Formula (10) with a compound of Formula (7):

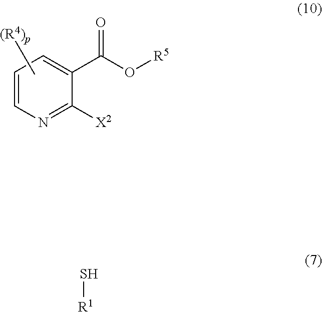

wherein the X² is hereinabove defined.

Such reactions may be carried out in a suitable solvent such as acetonitrile, butyronitrile or methanol for example, typically with the addition of a suitable base such as potassium carbonate or sodium hydroxide for example. Typically the reaction is carried out at elevated temperature, using Microwave or conventional heating, for example at temperatures between 100-140° C. In certain cases the reactions can be carried out at ambient temperature. Suitable examples of leaving groups for process (X²) are chloro, bromo, iodo, mesylate, tosylate or triflate. Others are known to the art.

Typically such reactions are carried out where R⁵ is an esterifying group, for example a methyl or an ethyl group which may be removed, for example, by hydrolysis with a base such as lithium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The reactions described above may be performed under standard conditions known to the person skilled in the art. The intermediates described above are commercially available, are known in the art or may be prepared by known procedures and/or by the procedures shown above.

It will be appreciated that certain of the various substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be to necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example hydroxylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess 11βHSD1 inhibitory activity. These properties may be assessed using the following assay.

### Assay

The conversion of cortisone to the active steroid cortisol by 11βHSD1 oxo-reductase activity, can be measured using a competitive homogeneous time resolved fluorescence assay (HTRF) (CisBio International, R&D, Administration and Europe Office, In Vitro Technologies—HTRF®/Bioassays BP 84175, 30204 Bagnols/Cèze Cedex, France. Cortisol bulk HTRF kit: Cat No. 62CORPEC).

The evaluation of compounds described herein was carried out using a baculovirus expressed N terminal 6-His tagged full length human 11βHSD1 enzyme(*1). The enzyme was purified from a detergent solublised cell lysate, using a copper chelate column. Inhibitors of 11βHSD1 reduce the conversion of cortisone to cortisol, which is identified by an increase in signal, in the above assay.

Compounds to be tested were dissolved in dimethyl sulphoxide (DMSO) to 10 mM and diluted further in assay buffer containing 10% DMSO to 10 fold the final assay concentration. Diluted compounds were then plated into black 384 well plates (Matrix, Hudson N.H., USA).

The assay was carried out in a total volume of 20 μl consisting of cortisone (Sigma, Poole, Dorset, UK, 160 nM), glucose-6-phosphate (Roche Diagnostics, 1 mM), NADPH (Roche Diagnostics, 100 μM), glucose-6-phosphate dehydrogenase (Roche Diagnostics, 12.5 μg/ml), EDTA (Sigma, Poole, Dorset, UK, 1 mM), assay buffer (K₂HPO₄/KH₂PO₄, 100 mM) pH 7.5, recombinant 11βHSD1 (1.5 μg/ml) plus test compound. The assay plates were incubated for 25 minutes at 37° C. after which time the reaction was stopped by the addition of 10 μl of 0.5 mM glycerrhetinic acid plus cortisol-XL665. 10 μl of anti-cortisol Cryptate was then added and the plates sealed and incubated for 6 hours at room temperature. Fluorescence at 665 nm and 620 nm was measured and the 665 nm:620 nm ratio calculated using an Envision plate reader.

This data was then used to calculate IC₅₀ values for each compound (Origin 7.5, Microcal software, Northampton Mass., USA).

### *1 The Journal of Biological Chemistry, Vol. 26, No 25, pp 16653-16658

Compounds of the present invention typically show an IC₅₀ of less than 30 μM, and preferably less than 5 μM.

For example, the following results were obtained:

| Example | IC₅₀ (μM) |
|---|---|
| 3 | 0.890 |
| 4 | 0.027 |

Compounds N-cyclohexyl-2-(phenethylsulfanyl)-6-trifluoromethylpyridine-3-carboxamide and N-cyclohexyl-2-[2-(2-carboxyphenyl)ethyl)sulfanyl]pyridine-3-carboxamide did not show activity in the above assay at 30 μM and as such are not a preferred aspect of the invention.

According to a further aspect of the invention there is provided a pharmaceutical composition, which comprises a compound of the Examples, or a pharmaceutically-acceptable salt thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). In general, compositions in a form suitable for oral use are preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl R-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

We have found that the compounds defined in the present invention, or a pharmaceutically-acceptable salt thereof, are effective 11βHSD1 inhibitors, and accordingly have value in the treatment of disease states associated with metabolic syndrome.

It is to be understood that where the term “metabolic syndrome” is used herein, this relates to metabolic syndrome as defined in 1) and/or 2) or any other recognised definition of this syndrome. Synonyms for “metabolic syndrome” used in the art include Reaven's Syndrome, Insulin Resistance Syndrome and Syndrome X. It is to be understood that where the term “metabolic syndrome” is used herein it also refers to Reaven's Syndrome, Insulin Resistance Syndrome and Syndrome X.

According to a further aspect of the present invention there is provided a compound of formula (1), or a pharmaceutically-acceptable salt thereof, as defined hereinbefore for use in a method of prophylactic or therapeutic treatment of a warm-blooded animal, such as man.

Thus according to this aspect of the invention there is provided a compound of formula (1), or a pharmaceutically-acceptable salt thereof, as defined hereinbefore for use as a medicament.

According to another feature of the invention there is provided the use of a compound of formula (1), or a pharmaceutically-acceptable salt thereof, as defined hereinbefore in the manufacture of a medicament for use in the production of an 11βHSD1 inhibitory effect in a warm-blooded animal, such as man.

Where production of or producing an 11βHSD1 inhibitory effect is referred to suitably this refers to the treatment of metabolic syndrome. Alternatively, where production of an 11βHSD1 inhibitory effect is referred to this refers to the treatment of diabetes, obesity, hyperlipidaemia, hyperglycaemia, hyperinsulinemia or hypertension, particularly diabetes and obesity. Alternatively, where production of an 11βHSD1 inhibitory effect is referred to this refers to the treatment of glaucoma, osteoporosis, tuberculosis, dementia, cognitive disorders or depression.

Alternatively, where production of an 11βHSD1 inhibitory effect is referred to this refers to the treatment of cognitive disorders, such as improving the cognitive ability of an individual, for example by improvement of verbal fluency, verbal memory or logical memory, or for treatment of mild cognitive disorders. See for example WO03/086410 and references contained therein, and Proceedings of National Academy of Sciences (PNAS), 2001, 98(8), 4717-4721.

Alternatively, where production of an 11βHSD1 inhibitory effect is referred to this refers to the treatment of, delaying the onset of and/or reducing the risk of atherosclerosis—see for example J. Experimental Medicine, 2005, 202(4), 517-527.

Alternatively, where production of an 11βHSD1 inhibitory effect is referred to this refers to the treatment of Alzheimers and/or neurodegenerative disorders.

According to a further feature of this aspect of the invention there is provided a method for producing an 11βHSD1 inhibitory effect in a warm-blooded animal, such as man, in need of such treatment which comprises administering to said animal an effective amount of a compound of formula (1), or a pharmaceutically-acceptable salt thereof.

In addition to their use in therapeutic medicine, the compounds of formula (1), or a pharmaceutically-salt thereof, are also useful as pharmacological tools in the development and standardisation of in vitro and in vivo test systems for the evaluation of the effects of inhibitors of 11βHSD1 in laboratory animals such as cats, dogs, rabbits, monkeys, rats and mice, as part of the search for new therapeutic agents.

The inhibition of 11βHSD1 described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets. For example agents than might be co-administered with 11βHSD1 inhibitors, particularly those of the present invention, may include the following main categories of treatment:

1) Insulin and insulin analogues;
2) Insulin secretagogues including sulphonylureas (for example glibenclamide, glipizide), prandial glucose regulators (for example repaglinide, nateglinide), glucagon-like peptide 1 agonist (GLP1 agonist) (for example exenatide, liraglutide) and dipeptidyl peptidase IV inhibitors (DPP-IV inhibitors);
3) Insulin sensitising agents including PPARγ agonists (for example pioglitazone and rosiglitazone);
4) Agents that suppress hepatic glucose output (for example metformin);
5) Agents designed to reduce the absorption of glucose from the intestine (for example acarbose);
6) Agents designed to treat the complications of prolonged hyperglycaemia; e.g. aldose reductase inhibitors
7) Other anti-diabetic agents including phosotyrosine phosphatase inhibitors, glucose 6-phosphatase inhibitors, glucagon receptor antagonists, glucokinase activators, glycogen phosphorylase inhibitors, fructose 1,6 bisphosphastase inhibitors, glutamine:fructose-6-phosphate amidotransferase inhibitors
8) Anti-obesity agents (for example sibutramine and orlistat);
9) Anti-dyslipidaemia agents such as, HMG-CoA reductase inhibitors (statins, eg pravastatin); PPARα agonists (fibrates, eg gemfibrozil); bile acid sequestrants (cholestyramine); cholesterol absorption inhibitors (plant stanols, synthetic inhibitors); ileal bile acid absorption inhibitors (IBATi), cholesterol ester transfer protein inhibitors and nicotinic acid and analogues (niacin and slow release formulations);
10) Antihypertensive agents such as, β blockers (eg atenolol, inderal); ACE inhibitors (eg lisinopril); calcium antagonists (eg. nifedipine); angiotensin receptor antagonists (eg candesartan), α antagonists and diuretic agents (eg. furosemide, benzthiazide);
11) Haemostasis modulators such as, antithrombotics, activators of fibrinolysis and antiplatelet agents; thrombin antagonists; factor Xa inhibitors; factor VIIa inhibitors; antiplatelet agents (eg. aspirin, clopidogrel); anticoagulants (heparin and Low molecular weight analogues, hirudin) and warfarin; and
12) Anti-inflammatory agents, such as non-steroidal anti-inflammatory drugs (eg. aspirin) and steroidal anti-inflammatory agents (eg. cortisone).

In the above other pharmaceutical composition, process, method, use and medicament manufacture features, the alternative and preferred embodiments of the compounds of the invention described herein also apply.

### EXAMPLES

The invention will now be illustrated by the following Examples in which, unless stated otherwise:

(i) temperatures are given in degrees Celsius (° C.); operations were carried out at room or ambient temperature, that is, at a temperature in the range of 18-25° C. and under an atmosphere of an inert gas such as argon;
(ii) evaporation of solvent was carried out using a rotary evaporator under reduced pressure (600-4000 Pa; 4.5-30 mmHg) with a bath temperature of up to 60° C.;
(iii) chromatography means flash chromatography on silica gel;
(iv) in general, the course of reactions was followed by TLC and reaction times are given for illustration only;
(v) yields are given for illustration only and are not necessarily those which can be obtained by diligent process development; preparations were repeated if more material was required;
(vi) where given, NMR data (¹H) is in the form of delta values for major diagnostic protons, given in parts per million (ppm) relative to tetramethylsilane (TMS), determined at 300 or 400 MHz (unless otherwise stated) using perdeuterio dimethyl sulfoxide (DMSO-d₆) as solvent, unless otherwise stated; peak multiplicities are shown thus: s, singlet; d, doublet; dd, doublet of doublets; dt, doublet of triplets; dm, doublet of multiplets; t, triplet, m, multiplet; br, broad;
(vii) chemical symbols have their usual meanings; SI units and symbols are used;
(viii) solvent ratios are given in volume: volume (v/v) terms;
(ix) mass spectra (MS) were run with an electron energy of 70 electron volts in the chemical ionisation (CI) mode using a direct exposure probe; where indicated ionisation was effected by electron impact (EI), fast atom bombardment (FAB) or electrospray (ESP); values for m/z are given; generally, only ions which indicate the parent mass are reported;
(x) The following abbreviations may be used below or in the process section hereinbefore:
Et₂O diethyl ether
DMF dimethylformamide
DCM dichloromethane
DME 1,2-dimethoxyethane
MeOH methanol
EtOH ethanol
H₂O water
TFA trifluoroacetic acid
THF tetrahydrofuran
DMSO dimethylsulfoxide
HOBt 1-hydroxybenzotriazole
EDCI (EDAC) 1-ethyl-3-(3-dimethylaminopropyl)carbodi-imide hydrochloride
DIPEA diisopropylethylamine
DEAD diethyl azodicarboxylate
EtOAc ethyl acetate
NaHCO₃ sodium bicarbonate
K₃PO₄ potassium phosphate
MgSO₄ magnesium sulfate
PS polymer supported
BINAP 2,2′-bis(diphenylphosphino)-1,1′binaphthyl
Dppf 1,1′-bis(diphenylphosphino)ferrocene dba dibenzylidineacetone
PS-CDI polymer supported carbonyldiimidazole

### Example 1

### N-Cyclohexyl-2-(3-phenylpropylsulfanyl)pyridine-3-carboxamide

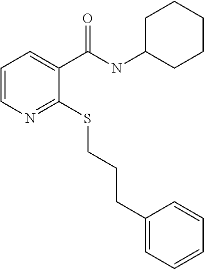

2-Chloro-N-cyclohexyl-pyridine-3-carboxamide (Intermediate 2, 0.84 mmol, 200 mg), 3-phenylpropane-1-thiol (2.0 mmol, 304 mg), and potassium carbonate (3.36 mmol, 128 mg) jn butyronitrile (3 mL) were heated at 130° C. for 30 minutes by microwaves.

The crude reaction mixture was filtered and placed directly onto reverse phase acidic HPLC using a Phenomenex Luna column 10 u C18(2) 100 A, 150×21.20 mm, and eluted with acetonitrile/water 0.2% TFA. The fractions containing N-cyclohexyl-2-(3-phenylpropylsulfanyl)pyridine-3-carboxamide were collected and concentrated in vacuo to give a white solid (72 mg, 25%).

¹H NMR (400.13 MHz, CDCl₃) δ1.11-1.22 (2H, m), 1.20-1.25 (1H, m), 1.28-1.34 (1H, m), 1.38-1.41 (1H, m), 1.50-1.60 (1H, m), 1.66-1.71 (2H, m), 1.93-2.00 (4H, m), 2.71 (2H, t), 3.20 (2H, t), 3.88-3.98 (1H, m), 6.36 (1H, d), 6.98-7.01 (1H, m), 7.12 (3H, t), 7.21 (1H, t), 7.74-7.77 (1H, m), 8.38-8.39 (1H, m);

MS m/e MH⁺ 355

### Example 2

### N-Cyclohexyl-2-(2-furylmethylsulfanyl)pyridine-3-carboxamide

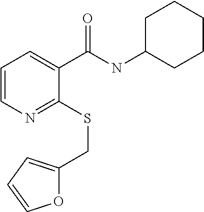

N-Cyclohexyl-2-(2-pyridin-2-ylethylsulfanyl)pyridine-3-carboxamide 2-chloro-N-cyclohexyl-pyridine-3-carboxamide (Intermediate 2, 0.42 mmol, 100 mg), 2-furylmethanethiol (2.0 mmol, 229 mg), and cesium carbonate (4.0 mmol, 546 mg) jn butyronitrile (3 mL) were heated at 150° C. for 30 minutes by microwaves. The crude reaction mixture was filtered and placed directly onto reverse phase acidic HPLC using a Phenomenex Luna column 10 u C18(2) 100 A, 150×21.20 mm, and eluted with acetonitrile/water 0.2% TFA. The fractions containing the product were collected and concentrated in vacuo to give the N-cyclohexyl-2-(2-furylmethylsulfanyl)pyridine-3-carboxamide (60 mg, 45%).

¹H NMR (400.13 MHz, CDCl₃) δ1.10-1.16 (1H, m), 1.20 (1H, d), 1.29-1.37 (2H, m), 1.54-1.59 (1H, m), 1.65-1.70 (2H, m), 1.93-1.95 (1H, m), 1.93-1.97 (2H, m), 2.02 (2H, d), 3.86-3.95 (1H, m), 4.48 (2H, s), 6.17-6.20 (2H, m), 6.20-6.24 (1H, m), 7.01-7.05 (1H, m), 7.19 (1H, s), 7.76-7.78 (1H, m), 8.44-8.46 (1H, m);

MS m/e MH⁺317

### Example 3

### N-Cyclohexyl-2-(2-pyridin-2-ylethylsulfanyl)pyridine-3-carboxamide

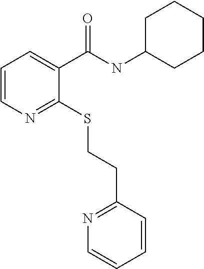

To an ice cooled solution of 2-(2-hydroxyethyl)pyridine (10.0 mmol, 1.23 g) in DCM (10 mL) was added triethylamine (12.0 mmol, 1.67 mL), followed by methane sulfonyl chloride (11.0 mmol, 0.85 mL).

The reaction was stirred at room temperature for 18 hours. Water (20 mL) was added and the mixture was extracted with DCM (2×30 mL). The organic phases were combined and washed successively with water (2×10 mL), brine (10 mL), dried over MgSO₄, filtered and evaporated under reduced pressure to give 2-(2-methylsulfonyloxyethyl)pyridine as a brown oil.

2-(2-methylsulfonyloxyethyl)pyridine (1.0 mmol, 201 mg), N-cyclohexyl-2-sulfanyl-pyridine-3-carboxamide (Intermediate 1, 1.0 mmol, 236 mg), and potassium carbonate (4.0 mmol, 152 mg) and acetonitrile (3 mL) were placed in a microwave vial. The reaction was heated by microwaves for 1 hour at 140° C. Water (10 mL) was added then the mixture was extracted with ethyl acetate (2×30 mL). The organic phases were combined and washed successively with water (2×10 mL), brine (10 mL), dried over MgSO₄, filtered and evaporated under reduced pressure. The oil obtained was purified by reverse phase basic HPLC using a C¹⁸ Silica Xtera column, 5 μm, 19×110 mm, acetonitrile/water 0.5% NH₃. The fractions containing the N-cyclohexyl-2-(2-pyridin-2-ylethylsulfanyl)pyridine-3-carboxamide were collected and concentrated in vacuo to give a white solid (167 mg, 49%).

¹H NMR (400.13 MHz, CDCl₃) δ1.21-1.21 (1H, m), 1.21 (2H, d), 1.33 (2H, d), 1.52 (1H, m), 1.63-1.68 (2H, m), 1.90-1.94 (2H, m), 3.16 (2H, t), 3.56 (2H, t), 3.86-3.95 (1H, m), 4.14 (1H, s), 6.46 (1H, d), 6.94-6.97 (1H, m), 7.09-7.12 (1H, m), 7.19 (1H, t), 7.56-7.61 (1H, m), 7.70-7.72 (1H, m), 8.36-8.38 (1H, m), 8.45 (1H, d);

MS m/e MH⁺ 342.

### Example 4

### N-Cyclohexyl-2-phenethylsulfanyl-pyridine-3-carboxamide

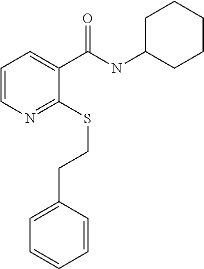

2-Chloro-N-cyclohexyl-pyridine-3-carboxamide (Intermediate 2, 0.42 mmol, 100 mg), 2-phenylethanethiol (0.84 mmol, 113 uL), and cesium carbonate (1.68 mmol, 546 mg) in butyronitrile (3 mL) were stirred together at room temperature for 18 hours.

The crude reaction mixture was filtered and placed directly onto reverse phase acidic HPLC using a Phenomenex Luna column 10 u C18(2) 100 A, 150×21.20 mm, and eluted with acetonitrile/water 0.2% TFA. The fractions containing N-cyclohexyl-2-phenethylsulfanyl-pyridine-3-carboxamide were collected and concentrated in vacuo to give a solid (79 mg, 55%).

¹H NMR (400.13 MHz, CDCl₃) δ1.10-1.18 (2H, m), 1.18-1.21 (1H, m), 1.29-1.37 (2H, m), 1.52-1.56 (1H, m), 1.63-1.69 (2H, m), 1.90-1.95 (2H, m), 2.93 (2H, t), 3.39-3.43 (2H, m), 3.87-3.96 (1H, m), 6.24-6.25 (1H, m), 6.96-6.99 (1H, m), 7.11-7.15 (1H, m), 7.17-7.24 (4H, m), 7.73-7.76 (1H, m), 8.40-8.42 (1H, m)

MS m/e MH⁺ 341.

### Example 5

### N-Cyclohexyl-2-(2-phenylpropylsulfanyl)pyridine-3-carboxamide

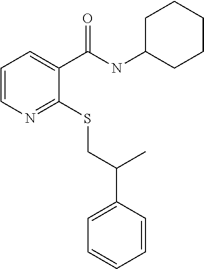

N-Cyclohexyl-2-sulfanyl-pyridine-3-carboxamide (Intermediate 1, 1.20 mmol, 284 mg), 1-bromo-2-phenylpropane (1.20 mmol, 239 mg), potassium carbonate (4.80 mmol, 183 mg) and acetonitrile (3 mL) were placed in a microwave vial. The reaction was heated by microwaves for 25 min at 130° C. The crude reaction mixture was filtered and placed directly onto a reverse phase basic C¹⁸ Silica Xtera column, 5 μm, 19×110 mm, and eluted with acetonitrile/water 0.5% NH₃. The fractions containing N-cyclohexyl-2-(2-phenylpropylsulfanyl)pyridine-3-carboxamide were collected and concentrated in vacuo to give a white solid (154 mg, 36%).

¹H NMR (400.13 MHz, DMSO) δ1.11 (1H, d), 1.21 (1H, s), 1.24-1.27 (2H, m), 1.31 (4H, d), 1.58 (1H, d), 1.71 (2H, d), 1.78-1.80 (2H, m), 2.99 (1H, q), 3.26-3.32 (1H, m), 3.67 (1H, d), 7.14-7.18 (1H, m), 7.22 (1H, d), 7.27-7.33 (4H, m), 7.65-7.67 (1H, m), 8.28 (1H, d), 8.49-8.51 (1H, m)

MS m/e MH⁺ 377.

### Example 6

### N-Cyclohexyl-5-fluoro-2-phenethylsulfanyl-pyridine-3-carboxamide

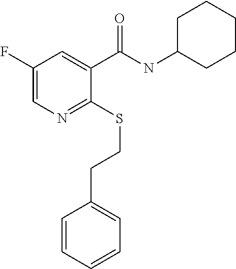

N-Cyclohexyl-5-fluoro-2-phenethylsulfanyl-pyridine-3-carboxamide was made in a similar manner to Example 5 from Intermediate 3 using butyronitrile as a solvent and heating for 30 minutes at 140° C. by microwaves.

¹H NMR (400.13 MHz, CDCl₃) δ1.16-1.22 (4H, m), 1.34 (1H, d), 1.37 (1H, s), 1.64-1.70 (0H, m), 1.93 (0H, s), 1.91-1.95 (1H, m), 2.92 (4H, d), 2.95 (1H, s), 3.45 (2H, t), 3.90-3.97 (1H, m), 6.45 (1H, d), 7.14-7.24 (7H, m), 7.62-7.65 (1H, m), 8.33 (1H, d);

MS m/e MH⁺ 359

### Example 7

### N-Cyclohexyl-2-phenacylsulfanyl-pyridine-3-carboxamide

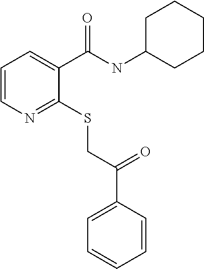

To a solution of N-cyclohexyl-2-sulfanyl-pyridine-3-carboxamide (Intermediate 1, 1.5 mmol, 354 mg) in THF (3 mL) was added a solution of 1N NaHMDS in THF (1.5 mmol, 1.5 mL). The reaction was stirred at room temperature for 5 minutes before adding bromoacetophenone (299 mg, 1.5 mmol). The reaction mixture was stirred at room temperature for one hour. LCMS showed that it had gone to completion. Water (10 mL) was added then the mixture was extracted with ethyl acetate (2×10 mL). The organic phases were combined and washed successively with water (2×10 mL), brine (10 mL), dried over MgSO₄, filtered and evaporated under reduced pressure. Purification by flash column chromatography (SiO₂, eluent gradient: 0% to 100% hexane:EtOAc) afforded the title compound (130 mg, 24%) as a white solid.

¹H NMR (400.13 MHz, CDCl₃) δ1.14-1.21 (1H, m), 1.25-1.33 (2H, m), 1.34-1.45 (2H, m), 1.51-1.59 (2H, m), 1.69-1.72 (2H, m), 1.98 (2H, d), 3.93-3.96 (1H, m), 4.57 (2H, s), 6.75 (1H, s), 6.99-7.02 (1H, m), 7.41 (2H, t), 7.49-7.53 (1H, m), 7.90 (3H, d), 8.24-8.26 (1H, m);

MS m/e MH⁺ 355.

### Example 8

### N-Cyclohexyl-2-(2-hydroxy-2-phenyl-ethyl)sulfanyl-pyridine-3-Carboxamide

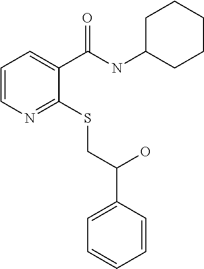

To a solution of N-cyclohexyl-2-phenacylsulfanyl-pyridine-3-carboxamide, Example 7, 0.34 mmol, 121 mg) in ethanol (5 mL) was added sodium borohydride (0.34 mmol, 13 mg). The reaction was stirred at room temperature for 30 minutes. LCMS showed that it had gone to completion. Water (10 mL) was added then the mixture was extracted with ethyl acetate (2×10 mL). The organic phases were combined and washed successively with water (10 mL), brine (10 mL), dried over MgSO₄, filtered and evaporated under reduced pressure. Purification by flash column chromatography (SiO₂, eluent gradient: 0% to 100% hexane:EtOAc) afforded the title compound (99 mg, 82%) as a gum.

¹H NMR (400.13 MHz, CDCl₃) δ1.06-1.25 (4H, m), 1.26-1.39 (1H, m), 1.28-1.40 (2H, m), 1.55 (1H, d), 1.89-1.93 (1H, m), 1.94 (2H, d), 3.32-3.36 (1H, m), 3.42-3.47 (1H, m), 3.83-3.93 (1H, m), 4.93-4.96 (1H, m), 6.29 (1H, d), 7.01-7.12 (1H, m), 7.15-7.23 (1H, m), 7.24-7.30 (2H, m), 7.35-7.39 (2H, m), 7.70-7.73 (1H, m), 8.35-8.36 (1H, m);

MS m/e MH⁺ 357.

### Example 9

### N-Cyclohexyl-2-[2-(4-methylsulfonyloxyphenyl)ethylsulfanyl]pyridine-3-carboxamide

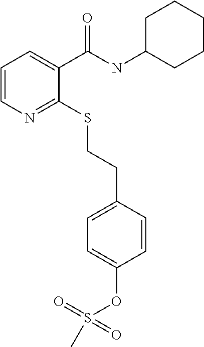

To an ice cooled solution of 4-hydroxyphenethyl alcohol (4.0 mmol, 552 mg) in DCM (10 mL) was added triethylamine (8.4 mmol, 1.169 mL), followed by methane sulfonyl chloride (8.4 mmol, 958 mg).

The reaction was stirred at room temperature for 18 hours. Water (20 mL) was added and the mixture was extracted with DCM (2×30 mL). The organic phases were combined and washed successively with water (2×10 mL), brine (10 mL), dried over MgSO₄, filtered and evaporated under reduced pressure to give 1-methylsulfonyloxy-4-(2-methylsulfonyloxyethyl)benzene as a yellow oil.

To a solution of N-cyclohexyl-2-sulfanyl-pyridine-3-carboxamide (Intermediate 1, 0.5 mmol, 118 mg) in THF (10 mL) was added a solution of 1N NaHMDS in THF (0.5 mmol, 0.5 mL). The reaction was stirred at room temperature for 5 minutes before adding the 1-methylsulfonyloxy-4-(2-methylsulfonyloxyethyl)benzene (0.5 mmol, 147 mg). The reaction was stirred at room temperature overnight. A solution of saturated bicarbonate (10 mL) was added then the mixture was extracted with ethyl acetate (2×30 mL). The organic phases were combined and washed successively with water (10 mL), brine (10 mL), dried over MgSO₄, filtered and evaporated under reduced pressure. The oil obtained was purified by reverse phase acidic HPLC using a Phenomenex Luna column 10 u C18(2) 100 A, 150×21.20 mm, and eluting with acetonitrile/water 0.2% TFA. The fractions containing N-cyclohexyl-2-[2-(4-methylsulfonyloxyphenyl)ethylsulfanyl]pyridine-3-carboxamide were collected and concentrated in vacuo to give a white solid (21 mg, 10%).

¹H NMR (400.13 MHz, CDCl₃) δ1.25 (2H, d), 1.31-1.39 (2H, m), 1.57-1.62 (1H, m), 1.69 (1H, d), 1.68-1.73 (1H, m), 1.93-1.97 (2H, m), 2.96 (2H, t), 3.08 (3H, s), 3.55 (2H, t), 3.91 (1H, t), 6.38 (1H, d), 7.05 (2H, d), 7.09-7.12 (1H, m), 7.20 (2H, d), 7.76-7.78 (1H, m), 8.45-8.47 (1H, m);

MS m/e MH⁺ 435.

### Example 10

### [3-(2-hydroxyethyl)-1-piperidyl]-(2-phenethylsulfanylpyridin-3-yl)methanone

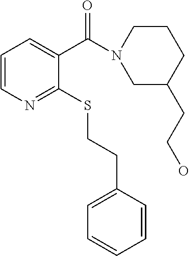

EDAC (1.5 mmol, 287 mg), HOBt (1.5 mmol, 203 mg), 2-phenethylsulfanylpyridine-3-carboxylic acid (1.5 mmol, 389 mg) and triethylamine (1.5 mmol, 0.209 mL) were stirred in DCM (10 mL). 2-(3-Piperidyl)ethanol was added and the reaction mixture was left to stir at room temperature overnight. Water (20 mL) was added and the mixture extracted with DCM (2×10 mL). The organic phases were combined, dried over MgSO₄, filtered and evaporated in vacuo. Purification by flash column chromatography (SiO₂, 0 to 100% hexane:ethyl acetate) afforded the title compound (143 mg, 26%) as a gum.

¹H NMR (400.13 MHz, CDCl₃) δ1.12-1.21 (1H, m), 1.25 (1H, d), 1.44-1.51 (2H, m), 1.68-1.69 (1H, m), 1.72 (1H, d), 1.78 (1H, d), 1.82 (1H, t), 2.32 (1H, s), 2.61 (1H, m), 2.90-2.92 (2H, t), 3.23 (1H, m), 3.38 (2H, t), 3.59-3.70 (1H, m), 4.37 (1H, m), 6.93-6.96 (1H, m), 7.10-7.14 (1H, m), 7.17-7.21 (3H, m), 7.19-7.23 (1H, m), 7.22 (0H, s), 7.31 (1H, d), 8.37-8.39 (1H, m);

MS m/e MH⁺ 371

### Example 11

### N-cyclohexyl-2-(2-pyridin-3-ylethylsulfanyl)pyridine-3-carboxamide

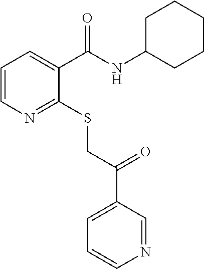

To a stirred solution of N-Cyclohexyl-2-sulfanyl-pyridine-3-carboxamide (Intermediate 1, 2.49 mmol, 588 mg), 2-(3-Pyridyl)Ethan-1-ol (2.49 mmol, 307 mg) and Triphenylphosphine (3.73 mmol, 978 mg) in anhydrous THF (40 ml) was added DIAD (3.73 mmol, 735 μl). The reaction was stirred at ambient temperature for 3 hours then partitioned between EtOAc (˜150 ml) and water (150 ml). The layers were separated and the organic layer was washed with 0.5N citric acid (˜100 ml), sat NaHCO₃ (˜100 ml) and brine (˜100 ml) then dried (MgSO₄), filtered and evaporated to a solid. This solid was purified by column chromatography (120 g Si, 20 to 60% EtOAc in Isohexane). Fractions containing product were evaporated to dryness to afford crude product which was recrystallised from EtOAc/IH to yield the title compound as a white solid (172 mg, 20%).

¹H NMR (300.073 MHz, DMSO-d₆) δ1.06-1.47 (5H, m), 1.55-1.98 (5H, m), 2.91 (2H, t), 3.35 (2H, t), 3.63-3.74 (1H, m), 7.15-7.19 (1H, m), 7.29-7.33 (1H, m), 7.67-7.71 (2H, m), 8.26 (1H, d), 8.40-8.42 (1H, m), 8.47-8.47 (1H, m), 8.51-8.53 (1H, m)

MS m/e MH⁺ 342.

### Example 12

### 6-Chloro-N-cyclohexyl-2-phenethylsulfanyl-pyridine-3-carboxamide

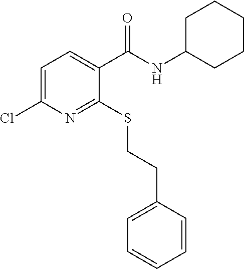

To a solution of 2-phenylethanethiol (2.2 mmol, 295 μl) in DMF (3 ml) was added NaHMDS (1M in THF) (2.2 mmol, 2.2 ml). The reaction was stirred at ambient temperature for 2 minutes then added to a solution of 2,6-dichloro-N-cyclohexyl-pyridine-3-carboxamide (Intermediate 4, 2.2 mmol, 600 mg) in DMF (2 ml). The reaction was stirred at ambient temperature for one hour then evaporated in vacuo. The resulting residue was partitioned between citric acid (˜20 ml) and EtOAc (˜40 ml). The layers were separated and the organic layer was washed with sat NaHCO₃ (˜20 ml), water (˜20 ml) and brine (˜10 ml), then dried (MgSO₄), filtered and evaporated to a solid. This solid was triturated with EtOAc/IH (˜1:9) to afford the title compound as a white solid (700 mg, 85%).

¹H NMR (400.13 MHz, DMSO-d₆) δ1.11-1.35 (5H, m), 1.57-1.60 (1H, m), 1.70-1.73 (2H, m), 1.81 (2H, m), 2.90 (2H, m), 3.25-3.29 (2H, m), 3.65-3.72 (1H, m), 7.20-7.27 (1H, m), 7.28-7.34 (5H, m), 7.79 (1H, d), 8.38 (1H, d)

MS m/e MH⁺ 375.

### Example 13

### N-Cyclohexyl-2-[2-(2-methylsulfonyloxyphenyl)ethylsulfanyl]pyridine-3-carboxamide

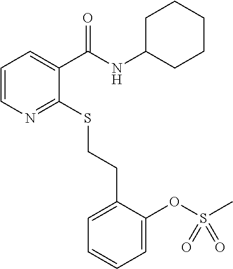

To a solution of 2-hydroxyphenethyl alcohol (14.48 mmol, 2 g) and MsCl (14.48 mmol, 5.60 ml) in DCM (50 ml) at 0° C. under Ar was slowly added triethylamine (14.48 mmol, 10.09 ml). The reaction was allowed to warm to ambient temperature and left to stir overnight. The reaction was diluted with DCM (˜30 ml) then washed with 1N citric acid (2×˜50 ml), sat NaHCO₃ (2×˜50 ml) and brine (˜50 mL). The organic solution was then dried (MgSO₄), filtered and evaporated to an oil. This oil was purified by column chromatography (40 g Si, eluting with 50 to 80% EtOAc in isohexane) to yield 1-methylsulfonyloxy-2-(2-methylsulfonyloxyethyl)benzene as a yellow oil (1.7 g, 40%).

A solution of N-cyclohexyl-2-sulfanyl-pyridine-3-carboxamide (Intermediate 1, 2.12 mmol, 500 mg) and 1-methylsulfonyloxy-2-(2-methylsulfonyloxyethyl)benzene (from above) (2.12 mmol, 623 mg) in EtOH (20 ml) was treated with Potassium carbonate (2.33 mmol, 322 mg) then warmed to reflux and stirred at this temperature overnight. The reaction mixture was evaporated under reduced pressure and the resulting residue partitioned between EtOAc (˜100 ml) and water (˜100 ml). The layers were separated and the organic layer was washed with water (˜50 ml) and brine (˜50 ml) then dried (MgSO₄), filtered and evaporated to a solid. This solid was recrystallised from EtOAc/IH to afford the title compound as a white solid (481 mg, 52%).

¹H NMR (300.073 MHz, DMSO-d₆) δ1.10-1.19 (1H, m), 1.22-1.36 (4H, m), 1.56-1.60 (1H, m), 1.69-1.74 (2H, m), 1.80-1.83 (2H, m), 3.00 (2H, t), 3.27-3.35 (2H, t), 3.43 (3H, s), 3.67-3.71 (1H, m), 7.15-7.19 (1H, m), 7.28-7.39 (3H, m), 7.44-7.48 (1H, m), 7.67-7.70 (1H, m), 8.26 (1H, d), 8.50-8.52 (1H, m)

MS m/e MH⁺ 435

### Example 14

### N-Cyclohexyl-2-[2-(2-hydroxyphenyl)ethylsulfanyl]pyridine-3-carboxamide

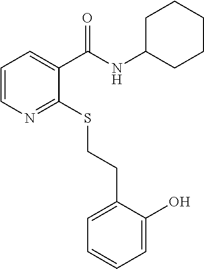

A solution of N-cyclohexyl-2-[2-(2-methylsulfonyloxyphenyl)ethylsulfanyl]pyridine-3-carboxamide (example 13) (0.71 mmol, 310 mg) in MeOH (14 ml)/2M NaOH (2 ml) was heated in a microwave at 140° C. for 30 minutes. 1 ml of 2M NaOH was added and the reaction was heated at 140° C. for a further 1 hour. The solvent level was reduced to ˜1/2 under reduced pressure then partitioned between EtOAc (˜80 ml) and water (˜80 ml). The layers were separated, the organic layer was washed with 0.5N citric acid (˜50 ml), water (˜50 ml) and brine (˜50 ml) then dried (MgSO₄), filtered and evaporated to a solid. This solid was purified by column chromatography (40 g Si, 20 to 60% EtOAc/1H) to afford the title compound as a white solid (9 mg, 4%).

¹H NMR (300.072 MHz, CDCl₃) δ1.13-1.52 (5H, m), 1.62-1.81 (3H, m), 2.03-2.08 (2H, m), 2.97-3.02 (2H, m), 3.19 (2H, m), 4.02 (1H, m), 6.09 (1H, d), 6.79-6.85 (1H, m), 6.94-6.97 (1H, m), 7.07-7.10 (1H, m), 7.15-7.21 (2H, m), 7.78-7.87 (1H, m), 8.58-8.60 (1H, m), 9.40 (1H, s)

MS m/e MH⁺ 357.

### Example 15

### 2-[2-(3-Carbamoylphenyl)ethylsulfanyl]-N-cyclohexyl-pyridine-3-carboxamide

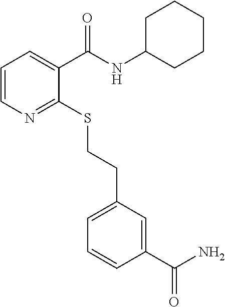

A solution of 2-[2-(3-cyanophenyl)ethylsulfanyl]-N-cyclohexyl-pyridine-3-carboxamide Example 32, 1.18 mmol, 430 mg) in CH₂SO₄ (1.8 ml) was stirred at 80° C. for 30 minutes. The reaction was cooled to ambient temperature, cautiously poured into ice water then treated with sat NaHCO₃ until basic. The aqueous solution was extracted with EtOAc (3×150 ml). The combined organic layers were washed with brine (˜50 ml), dried (MgSO₄), filtered and evaporated to a solid. This solid was triturated with ether (˜30 ml), filtered and dried to afford the title compound as a white solid (404 mg, 90%).

¹H NMR (400.13 MHz, DMSO-d₆) δ1.08-1.18 (1H, m), 1.21-1.36 (4H, m), 1.57-1.60 (1H, m), 1.68-1.74 (2H, m), 1.81-1.84 (2H, m), 2.96 (2H, t), 3.38 (2H, t), 3.66-3.74 (1H, m), 7.16-7.19 (1H, m), 7.29 (1H, s), 7.38 (1H, m), 7.42-7.44 (1H, m), 7.68-7.70 (1H, m), 7.71-7.74 (1H, m), 7.78 (1H, s), 7.91 (1H, s), 8.25 (1H, d), 8.52-8.54 (1H, m)

MS m/e MH⁺ 384.

### Example 16

### 3-[2-[3-(Cyclohexylcarbamoyl)pyridin-2-yl]sulfanylethyl]benzoic acid

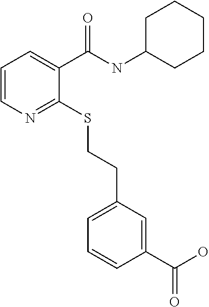

cHCl (11 ml) was added to a stirred suspension of 2-[2-(3-cyanophenyl)ethylsulfanyl]-N-cyclohexyl-pyridine-3-carboxamide (Example 32, 0.62 mmol, 289 mg) in water (6 ml). The resulting solution was heated in a microwave at 140° C. for 10 minutes. The reaction mixture was cautiously added to satNaHCO₃ solution then the resulting suspension was cautiously treated with citric acid until ˜pH4. The mixture was then extracted with EtOAc (3×˜150 ml). The organic layers were combined, washed with brine (˜50 ml) then dried (MgSO₄), filtered and evaporated to a solid. This solid was recrystallised from MeOH to yield the title compound as a white solid (184 mg, 64%).

¹H NMR (400.13 MHz, DMSO-d₆) δ1.15 (1H, m), 1.22-1.36 (4H, m), 1.59 (1H, d), 1.71-1.74 (2H, m), 1.82 (2H, m), 2.99 (2H, t), 3.39 (2H, t), 3.66-3.73 (1H, m), 7.16-7.19 (1H, m), 7.43 (1H, t), 7.54 (1H, d), 7.68-7.70 (1H, m), 7.81 (1H, d), 7.86 (1H, s), 8.23 (1H, d), 8.52-8.54 (1H, m), 12.87 (1H, s)

MS m/e M−H⁻ 383.

### Example 17

### Cis-4-[(6-methyl-2-phenethylsulfanyl-pyridine-3-carbonyl)amino]cyclohexane-1-carboxylic acid

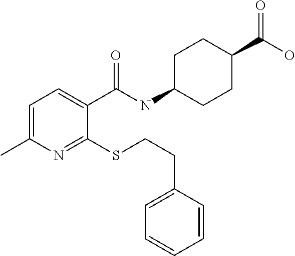

Sodium-bis-trimethylsilylamide (1.5 ml) was added to benzethanthiol (207 mg. 1.5 mmol) in DMF (2 ml). The resulting yellow suspension was added to a stirred solution of methyl cis-4-{[(2-chloro-6-methylpyridin-3-yl)carbonyl]amino}cyclohexanecarboxylate (Intermediate 7, 393 mg, 1-0.26 mmol) in DMF (10 ml) and stirred at room temperature for 4 hours. The reaction was diluted with water (100 ml) and extracted with dichloromethane (2×50 ml), the organic phase was washed with 1M HCl (25 ml), sat.NaHCO₃ soln. (25 ml), water (25 ml), brine (25 ml), dried over MgSO₄ filtered and the solvent was removed in vacuo Chromatography SiO2 (40 g) eluting with ethyl acetate/isohexane 10-60% gave methyl Cis-4-[(6-methyl-2-phenethylsulfanyl-pyridine-3-carbonyl)amino]cyclohexane-1-carboxylate as a clear oil (298 mg, 57%). This was dissolved in MeOH (5 ml) and 2M NaOH (2 ml) was added and stirred for 16 hours at room temperature. The methanol was removed in vacuo and the solution pH adjusted to 3 with c.HCl. The resultant precipitate was extracted into dichloromethane (25 ml) dried over MgSO₄ filtered and the solvent removed in vacuo to give a gum which on trituration with ether/isohexane gave the title compound (185 mg, 64%) as a white powder.

¹H NMR (400.13 MHz, DMSO-d₆) δ1.59 (6H, m), 1.95 (2H, m), 2.43 (1H, s), 2.52 (3H, s), 2.88 (2H, m), 3.27-3.31 (2H, m), 3.82 (1H, s), 7.03 (1H, d), 7.20-7.24 (1H, m), 7.30 (4H, m), 7.62 (1H, m), 8.25 (1H, d), 12.14 (1H, s)

MS m/e (M+H)=399.

### Example 18

### 4-[Methyl-(6-methyl-2-phenethylsulfanyl-pyridine-3-carbonyl)amino]cyclohexane-1-carboxylic acid

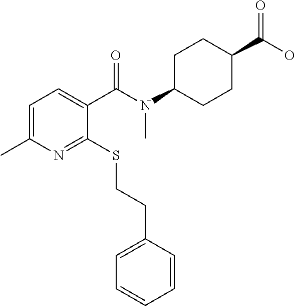

To cis-4-({[2-(phenethythio)-6-methylpyridin-3-yl]carbonyl}amino)cyclohexanecarboxylic acid (Example 18, 190 mg, 0.48 mmol) stirred in THF (5 ml) under nitrogen was added drop wise LHMDS 1.0M in THF (1.1 ml, 1.1 mmol) and the reaction stirred for a further 5 minutes. Iodmethane (33 ul, 0.52 mmol) in THF (1 ml) was added drop wise and stirred for 16 hours at room temperature. The reaction was diluted with dichloromethane (50 ml) and washed with 1MHCl (25 ml), 10% thiosulphate (25 ml), water (25 ml), brine (25 ml), dried over MgSO₄, filtered and evaporated. The resulting gum was purified by RPHPLC Phenomenex Luna C¹⁸, 10 μl 150×21.1 mm eluting with acetonitrile 0.2% TFA/water0.2% TFA 45-65% to give the title compound (90 mg, 45%) as a yellow foam.

¹H NMR (400.13 MHz, DMSO-d₆) δ1.45 (2H, m), 1.59 (3H, m), 1.70-1.74 (3H, m), 2.11 (2H, d), 2.54 (3H, s), 2.70 (3H, s), 2.95 (2H, t), 3.45 (2H, m), 7.00 (1H, d), 7.20 (1H, d), 7.26-7.29 (4H, m), 7.36 (1H, d), 12.12 (1H, bs)

MS m/e=(M+H)=413.

### Example 19

### 6-Chloro-N-[(2r,5s)-5-hydroxy-2-adamantyl]-2-phenethylsulfanyl-pyridine-3-carboxamide

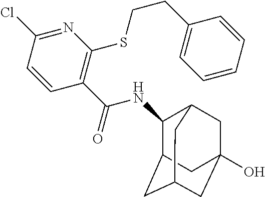

2,6-Dichloro-N-[(2r,5s)-5-hydroxy-2-adamantyl]pyridine-3-carboxamide (Intermediate 8, 1.17 mmol, 400 mg), 2-phenylethanethiol (1.17 mmol, 162 mg) and sodium carbonate (3.51 mmol, 373 mg) in DMF (5 ml) were stirred at room temperature for 18 hours. Water (20 mL) was added then the mixture was extracted with ethyl acetate (2×50 μL). The organic phases were combined and washed successively with water (2×20 mL), brine (10 mL), dried over MgSO₄, filtered and evaporated under reduced pressure. Purification by flash column chromatography (SiO₂, eluent gradient: 0% to 100% DCM:EtOAc) afforded the title compound (505 mg, 97%) as a white solid.

¹H NMR (400.13 MHz, DMSO-d₆) δ1.29-1.37 (2H, m), 1.60-1.66 (4H, m), 1.68-1.75 (2H, m), 1.91-2.08 (5H, m), 2.89-2.93 (2H, m), 3.27-3.33 (2H, m), 3.87-3.92 (1H, m), 4.46 (1H, s), 7.20-7.26 (1H, m), 7.30-7.34 (5H, m), 7.77 (1H, d), 8.30 (1H, d)

MS m/e MH⁺ 443

### Example 20

### (1r,4s)-4-[([6-Methyl-2-[(2-phenylethyl)thio]pyridin-3-yl]carbonyl)amino]adamantane-1-carboxylic acid

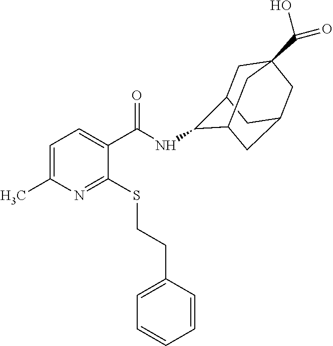

Lithium hydroxide monohydrate (0.176 g, 4.19 mmol) was added in one portion to methyl (1r,4s)-4-[({6-methyl-2-[(2-phenylethyl)thio]pyridin-3-yl}carbonyl)amino]adamantane-1-carboxylate (Intermediate 10, 389 mg, 0.84 mmol) in THF:water 4:1 (10 mL). The resulting solution was stirred at room temperature for 7 days. The reaction mixture was evaporated to dryness and redissolved in water (10 mL) and adjusted to pH=3 with 2M HCl. The precipitate was collected by filtration, washed with water (20 mL) and dried under vacuum to afford the title compound (357 mg, 95%) as a white solid.

m/z (ESI+) (MH⁺)=451

¹H NMR (400.13 MHz, DMSO-d₆) δ 1.45 (2H, d), 1.79 (2H, t), 1.90 (5H, s), 2.06 (4H, d), 2.52 (3H, s), 2.91 (2H, m), 3.32-3.35 (2H, m), 3.94 (1H, d), 7.04 (1H, d), 7.20-7.24 (1H, m), 7.28-7.33 (4H, m), 7.63 (1H, d), 8.13 (1H, d), 12.02 (1H, s)

### Reference Example 1

### N-Cyclohexyl-2-[[3-(trifluoromethyl)phenyl]methylsulfanyl]pyridine-3-carboxamide

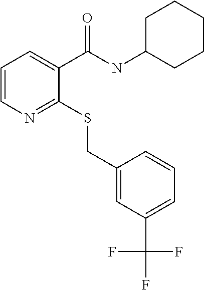

2-Chloro-N-cyclohexyl-pyridine-3-carboxamide (Intermediate 2, 0.42 mmol, 100 mg), [3-(trifluoromethyl)phenyl]methanethiol (0.84 mmol, 162 mg), and cesium carbonate (4.0 mmol, 546 mg) jn butyronitrile (3 mL) were stirred at room temperature for 18 hours.

The crude reaction mixture was filtered and placed directly onto reverse phase acidic HPLC using a Phenomenex Luna column 10 u C18(2) 100 A, 150×21.20 mm, and eluted with acetonitrile/water 0.2% TFA. The fractions containing the purified compound were collected and concentrated in vacuo to give the product (55 mg, 33%).

¹H NMR (400.13 MHz, CDCl₃) δ1.12-1.22 (2H, m), 1.25 (1H, s), 1.31-1.39 (2H, m), 1.66-1.71 (2H, m), 1.95-1.99 (2H, m), 3.21 (1H, s), 3.89-3.98 (1H, m), 6.30-6.31 (1H, m), 7.04-7.08 (1H, m), 7.14 (1H, t), 7.42 (2H, d), 7.79-7.82 (1H, m), 8.30-8.31 (1H, m);

MS m/e MH⁺ 395.

### Intermediate 1

### N-cyclohexyl-2-sulfanyl-pyridine-3-carboxamide

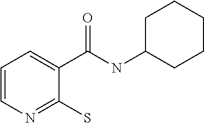

2-sulfanylpyridine-3-carboxylic acid (3.88 g, 25.0 mmol), cyclohexylamine (2.855 mL, 25.0 mmol), Et₃N (6.96 mL, 50.0 mmol) and HOBT (3.38 g, 25.0 mmol) were dissolved in DCM (40 mL). EDAC (5.255 g, 27.50 mmol) was added and the reaction stirred at ambient temperature for 19 h. Water (50 mL) was added then the mixture was extracted with DCM (2×30 mL). The organic phases were combined and washed successively with sat. NaHCO₃ (30 mL), water (2×30 mL), brine (30 mL), dried over MgSO₄, filtered and evaporated under reduced pressure. Purification by flash column chromatography (SiO₂, 0 to 10% MeOH in DCM gradient) afforded the title compound (5.071 g, 52%) as a yellow solid.

¹H NMR (400.13 MHz, DMSO-d₆) δ1.24 (1H, s), 1.33 (2H, d), 1.38 (5H, t), 1.52 (1H, s), 1.68 (1H, s), 1.69 (3H, t), 1.82 (2H, d), 3.88 (1H, s), 7.02-7.06 (1H, m), 7.96-7.97 (1H, m), 8.52-8.54 (1H, m), 10.94 (1H, d);

MS m/e MH⁺ 237.

### Intermediate 2

### 2-chloro-N-cyclohexyl-pyridine-3-carboxamide

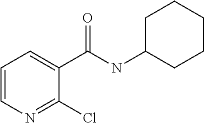

To a solution of 2-chloro nicotinic acid (9.45 g, 60.0 mmol) in DCM (50 mL) was added carefully with stirring thionyl chloride (15.325 mL, 210.0 mmol, 3.5 eq). After addition, DMF (1 mL) was added carefully and the reaction mixture was stirred under reflux for three hours.

The reaction was allowed to cool down to room temperature and the thionyl chloride in excess was taken off on a rotary evaporator. The product was further dried under high vacuum.

The 2-chloropyridine-3-carbonyl chloride (60.0 mmol) obtained was dissolved in dichloromethane (20 mL) and added to an ice cooled solution of cyclohexylamine (6.855 mL, 60.0 mmol) and triethylamine (16.7 mL, 120.0 mmol, 2 eq) in DCM (20 mL). The reaction was stirred at room temperature for approximately 18 hours. Water (40 mL) was added and the mixture extracted with DCM (2×50 mL). The organic phases were combined, dried over MgSO₄, filtered and evaporated in vacuo. Purification by flash column chromatography (SiO₂, 30 to 100% ethyl acetate/hexane) afforded the title compound (10.0 g, 70%) as a white solid.

¹H NMR (400.13 MHz, DMSO-d₆) δ1.10-1.38 (5H, m), 1.55 (2H, d), 1.71-1.75 (2H, m), 1.85 (2H, d), 3.70-3.78 (1H, m), 7.46-7.49 (1H, m), 7.83-7.85 (1H, m), 8.45 (2H, m);

MS m/e MH⁺ 239.

### Intermediate 3

### 2-chloro-N-cyclohexyl-5-fluoro-pyridine-3-carboxamide

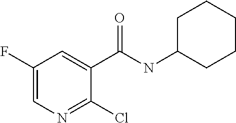

2-Chloro-5-fluoro Nicotinic acid (1.93 g, 11.0 mmol), cyclohexylamine (1.26 mL, 11.0 mmol), Et₃N (1.535 mL, 11.0 mmol) and HOBT (1.49, 11 mmol) were dissolved in DCM (30 mL). EDAC (2.105 g, 11.0 mmol) was added and the reaction was stirred at ambient temperature for 19 h.

DCM was added (20 mL) and the organic phase was washed with water (2×15 mL), brine (15 mL), dried (MgSO₄) and the solvent removed in vacuo. Purification by flash column chromatography (SiO₂, 0 to 10% MeOH in DCM gradient) afforded the title compound as a white solid (1.1 g, 38%).

¹H NMR (400.13 MHz, CDCl₃) δ1.13-1.22 (1H, m), 1.26 (2H, d), 1.33-1.40 (2H, m), 1.54-1.61 (1H, m), 1.66-1.72 (2H, m), 1.95-1.99 (2H, m), 3.89-3.98 (1H, m), 6.35 (1H, s), 7.79-7.81 (1H, m), 8.24 (1H, d);

MS m/e MH⁺ 257.

### Intermediate 4

### 2,6-dichloro-N-cyclohexyl-pyridine-3-carboxamide

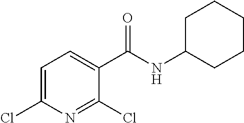

To a stirred suspension of 2,6-dichloronicotinic acid (79 mmol, 15 g), in DCM (120 ml) was added a few drops of DMF followed by dropwise addition of oxalyl chloride (87 mmol, 7.5 ml). The reaction was stirred at ambient temperature for 2.5 hours then evaporated in vacuo. The resulting oil was azeotroped with toluene then redissolved in DCM (100 ml). This solution was cooled to 0° C. and treated with a dropwise addition of cyclohexylamine (157 mmol, 17.9 ml). The reaction was allowed to warm to ambient temperature and stirred at this temperature for 1 hour. The reaction was diluted with DCM (˜50 ml), washed with sat NaHCO₃ (˜200 ml), water (˜200 ml) and brine (˜50 ml) then dried (MgSO₄), filtered and evaporated to an orange/brown solid. This material was triturated with IH/EtOAc (˜9:1) to afford the title compound as a white solid (20.36 g, 95%).

¹H NMR (300.073 MHz, DMSO-d₆) δ1.07-1.38 (5H, m), 1.54-1.58 (1H, m), 1.68-1.73 (2H, m), 1.81-2.52 (2H, m), 3.66-3.77 (1H, m), 7.60-7.64 (1H, d), 7.92 (1H, d), 8.47 (1H, d)

MS m/e [M+CH₃CN]H⁺ 314.

### Intermediate 5

### 2-(2-hydroxyethyl)benzonitrile

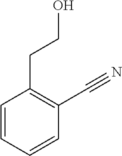

A dry 50 ml flask was charged with KCN (36 mmol, 2.34 g), acetonitrile (23 ml) and 2-bomophenethyl alcohol (24 mmol, 3.26 ml). The suspension was degassed three times (vacuum/Nitrogen), and then tibutyltin chloride (0.02 mmol, 5 μl), Pd2(dba)3 (0.12 mmol, 110 mg) and t-Bu3P (10% weight in hexanes, 0.6 mmol, 1.89 ml) were added. The suspension was degassed three times and stirred at ambient temperature for 30 minutes. The mixture was then degassed once more and then heated at 80° C. overnight. The reaction mixture was diluted with EtOAc (˜100 ml) and washed with water (2×˜100 ml) and brine (˜50 ml) then dried (MgSO4), filtered and evaporated to an oil. This oil was purified by column chromatography (120 g Si, 20 to 40% EtOAc in IH) to afford the title compound as a yellow oil (1.4 g, 40%).

¹H NMR (300.073 MHz, DMSO-d₆) δ2.93 (2H, t), 3.63-3.69 (2H, q), 4.79 (1H, t), 7.36-7.42 (1H, m), 7.47-7.50 (1H, m), 7.59-7.65 (1H, m), 7.74-7.77 (1H, m)

### Intermediate 6

### 3-(2-hydroxyethyl)benzonitrile

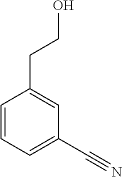

Compound made using method described above, replacing 2-bromophenethyl alcohol with 3-bromophenethyl alcohol

¹H NMR (300.072 MHz, CDCl₃) δ1.51 (1H, s), 2.91 (2H, t), 3.90 (2H, t), 7.39-7.55 (4H, m)

### Intermediate 7

### Methyl cis-4-{[(2-chloro-6-methylpyridin-3-yl)carbonyl]amino}cyclohexanecarboxylate

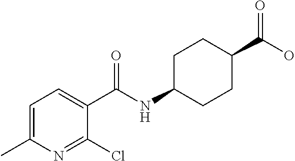

To a stirred solution of 2-chloro-6-methyl-nicotinic acid (566 mg, 3.3 mmol) in dichloromethane (25 ml) was added, HOBt (491 mg, 3.61 mmol), triethylamine (1.38 ml, 9.9 mmol) and EDAC (757 mg, 3.96 mmol). After 5 minutes methyl-cis-4-aminocyclohexanecarboxylate (639 mg, 3.3 mmol) was added and allowed to stir at room temperature for 16 hours. The reaction was diluted with dichloromethane (100 ml), washed with sat NaHCO₃ (50 ml), 1M HCl (50 ml), water (50 ml), brine (50 ml), dried over MgSO₄, filtered and the solvent removed in vacuo. Chromatography SiO₂ (40 g) eluting with ethyl acetate/isohexane 0-80% gave the title compound (787 mg, 76%) as a white solid.

¹H NMR (400.13 MHz, DMSO-d₆) δ1.56-1.69 (6H, m), 1.85-1.92 (2H, m), 2.48 (3H, s), 3.61 (3H, s), 3.90 (1H, t), 7.31 (1H, d), 7.72 (1H, d), 8.46 (1H, d)

MS m/e=(M+H)=311.

### Intermediate 8

### 2,6-dichloro-N-[(2r,5s)-5-hydroxy-2-adamantyl]pyridine-3-carboxamide

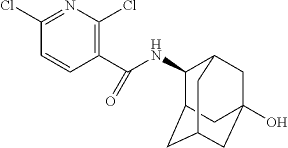

2,6-Dichloronicotinoyl chloride (48 mmol, 9.22 g) in DCM (40 mL) was added to 4-aminoadamantan-1-ol (48 mmol, 8.03 g) and Diisopropylethylamine (57.6 mmol, 7.44 g) in anhydrous THF (160 mL) at 0° C. under inert atmosphere. The mixture was allowed to warm to ambient temperature then stirred for 18 hours. Water (20 mL) was added then the mixture was extracted with ethyl acetate (2×250 mL). The organic phases were combined and washed successively with water (2×50 mL), brine (50 mL), dried over MgSO₄, filtered and evaporated under reduced pressure. Purification by flash column chromatography (SiO₂, eluent gradient: 0% to 100% DCM:EtOAc) afforded the title compound (9.37 g, 57%) as a white solid.

¹H NMR (400.13 MHz, DMSO-d₆) δ1.32-1.39 (2H, m), 1.61-1.68 (4H, m), 1.70-1.76 (2H, m), 1.88-1.95 (2H, m), 1.98-2.03 (1H, m), 2.05-2.10 (2H, m), 3.92-3.97 (1H, m), 4.41 (1H, s), 7.63 (1H, d), 7.93 (1H, d), 8.44 (1H, d)

MS m/e MH⁺ 341

### Intermediate 9

### methyl (1r,4s)-4-{[(2-chloro-6-methylpyridin-3-yl)carbonyl]amino}adamantane-1-carboxylate

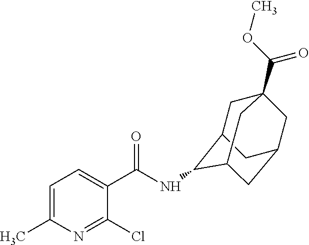

A solution of 2-chloro-6-methylnicotinoyl chloride (2.090 g, 11 mmol) in DCM (20 mL) was added to a stirred solution of methyl 4-aminoadamantane-1-carboxylate hydrochloride (2.70 g, 11.00 mmol), and N-ethyldiisopropylamine (9.52 mL, 55.00 mmol) in DCM (50 mL) at 20° C., over a period of 5 minutes under nitrogen. The resulting suspension was stirred for 16 hours. The reaction mixture was diluted with water (50 mL), and washed to sequentially with 1M HCl (25 mL), saturated NaHCO₃ (25 mL), and saturated brine (25 mL). The organic layer was dried over MgSO₄, filtered and evaporated to afford crude product. The crude gum was triturated with Et₂O to give a solid that was collected by filtration and dried under vacuum to give the title compound (2.90 g, 72.7%) as a white solid.

¹H NMR (400.13 MHz, DMSO-d₆) δ 1.46 (2H, d), 1.80 (4H, d), 1.92-1.96 (4H, m), 2.02-2.16 (3H, m), 2.48 (3H, s), 3.59 (3H, d), 3.97 (1H, d), 7.31-7.34 (1H, m), 7.73 (1H, t), 8.47-8.53 (1H, m)

m/z (ESI+) (MH⁺)=363.

### Intermediate 10

### methyl (1r,4s)-4-[({6-methyl-2-[(2-phenylethyl)thio]pyridin-3-yl}carbonyl)amino]adamantane-1-carboxylate

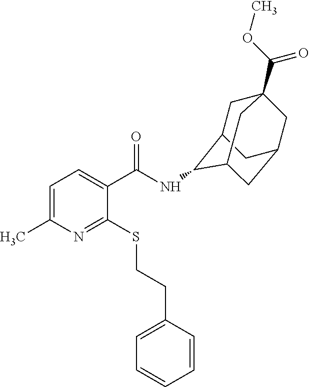

A solution of methyl (1r,4s)-4-{[(2-chloro-6-methylpyridin-3-yl)carbonyl]amino}adamantane-1-carboxylate (Intermediate 9, 363 mg, 1.00 mmol) in THF (5.00 ml) was added to a stirred suspension of 2-phenylethanethiol (0.134 ml, 1.00 mmol), and sodium bis(trimethylsilyl)amide (1 ml, 1.00 mmol) in THF/DMF9:1 (10.00 ml) at room temperature, over a period of 2 minutes under nitrogen. The resulting solution was stirred at room temperature for 2 hours. The reaction mixture was diluted with 50% brine (25 mL), and washed twice with EtOAc (25 mL). The organic layer was dried over MgSO₄, filtered and evaporated to afford crude product. The crude product was purified by flash silica (40 g) chromatography, elution gradient 20 to 50% EtOAc in isohexane. Pure fractions were evaporated to dryness to afford the title compound (389 mg, 84%) as a white foam.

m/z (ESI+) (MH⁺)=465

¹H NMR (400.13 MHz, DMSO-d₆) δ 1.45 (2H, d), 1.83 (2H, d), 1.92-1.93 (6H, m), 2.05-2.08 (4H, m), 2.52 (3H, s), 2.90 (2H, m), 3.33-3.61 (4H, m), 3.94-3.95 (1H, m), 7.04 (1H, d), 7.20-7.24 (1H, m), 7.28-7.32 (4H, m), 7.62-7.64 (1H, m), 8.15 (1H, d)

The following examples in the table below were prepared according to the general methods outlined in the examples above and from the appropriate starting materials and the intermediates described.

| | Method | | | | |
|---|---|---|---|---|---|
| | (Example | | | | MS m/e |
| Example No. | No.) | Intermediate | Name | ¹H NMR δ (CDCl₃) | MH⁺ |
| 21 | 1 | 1 | N-cyclohexyl-2-[2- | 1.12-1.25 (3H, m), 1.36-1.45 (2H, | 409 |
| | | | [3- | m), 1.55-1.60 (1H, m), | |
| | | | (trifluoromethyl)phenyl]ethylsulfanyl]pyridine- | 1.65-1.71 (2H, m), 1.92-1.96 (2H, m), 3.03 (2H, | |
| | | | 3- | t), 3.57 (2H, t), 3.88-3.95 (1H, m), | |
| | | | carboxamide | 6.34 (1H, d), 7.12-7.15 (1H, m), | |
| | | | | 7.39-7.45 (4H, m), 7.87 (1H, d), 8.47 (1H, d) | |
| 22 | 5 | 1 | N-cyclohexyl-2-[2- | 1.12-1.16 (1H, m), 1.17-1.20 (1H, | 359 |
| | | | (4- | m), 1.21 (1H, d), 1.34-1.40 (1H, m), | |
| | | | fluorophenyl)ethylsulfanyl]pyridine- | 1.57-1.60 (1H, m), 1.65-1.71 (2H, | |
| | | | 3- | m), 1.92-1.96 (2H, m), 2.92 (2H, t), | |
| | | | carboxamide | 3.48 (2H, t), 3.87-3.96 (1H, m), | |
| | | | | 6.40 (1H, d), 6.87-6.93 (2H, m), | |
| | | | | 7.07-7.11 (1H, m), 7.13-7.16 (1H, m), 7.19 (1H, | |
| | | | | s), 7.82-7.85 (1H, m), 8.42-8.44 (1H, | |
| | | | | m) | |
| 23 | 5 | 1 | 2-[2-(4- | (DMSO) 1.13 (1H, d), 1.27 (3H, d), | 375 |
| | | | chlorophenyl)ethylsulfanyl]- | 1.33 (1H, d), 1.59 (1H, d), 1.72 (2H, d), | |
| | | | N- | 1.80-1.82 (2H, m), 2.90 (2H, d), | |
| | | | cyclohexyl- | 3.66-3.72 (1H, m), 7.17-7.20 (1H, m), | |
| | | | pyridine-3- | 7.29-7.31 (2H, m), 7.34-7.37 (2H, m), | |
| | | | carboxamide | 7.68-7.71 (1H, m), 8.31 (1H, d), | |
| | | | | 8.52-8.54 (1H, m) | |
| 24 | 5 | 1 | N-cyclohexyl-2-(1- | (DMSO) 1.22 (4H, d), 1.28 (3H, d), | 355 |
| | | | phenylpropan-2- | 1.34 (1H, d), 1.59 (1H, d), 1.73 (2H, d), | |
| | | | ylsulfanyl)pyridine- | 1.81-1.84 (2H, m), 2.69-2.74 (1H, | |
| | | | 3-carboxamide | m), 3.00-3.05 (1H, m), 3.69 (1H, q), | |
| | | | | 4.07-4.16 (1H, m), 7.15-7.18 (1H, | |
| | | | | m), 7.20-7.24 (1H, m), | |
| | | | | 7.26-7.33 (4H, m), 7.65-7.68 (1H, m), 8.30 (1H, | |
| | | | | d), 8.49-8.55 (1H, m) | |
| 25 | 3 | 1 | N-cyclohexyl-2-(2- | 1.10-1.20 (2H, m), 1.23 (1H, s), | 342 |
| | | | pyridin-4- | 1.33-1.39 (1H, m), 1.48-1.59 (1H, m), | |
| | | | ylethylsulfanyl)pyridine- | 1.64-1.70 (2H, m), 1.94 (1H, d), | |
| | | | 3- | 1.92-1.96 (2H, m), 2.96 (2H, t), 3.37-3.40 (2H, | |
| | | | carboxamide | t), 3.69 (1H, s), 3.86-3.95 (1H, m), | |
| | | | | 6.36 (1H, d), 6.94-6.97 (1H, m), | |
| | | | | 7.18-7.21 (1H, m), 7.58 (1H, d), | |
| | | | | 7.67-7.70 (1H, m), 8.37-8.38 (2H, m), 8.42 (1H, | |
| | | | | s) | |
| 26 | 3 | 1 | N-cyclohexyl-2-[2- | 1.19 (1H, d), 1.21-1.24 (1H, m), | 357 |
| | | | (4- | 1.32-1.38 (1H, m), 1.53-1.58 (1H, m), | |
| | | | hydroxyphenyl)ethylsulfanyl]pyridine- | 1.64-1.70 (2H, m), 1.91-1.95 (2H, m), | |
| | | | 3-carboxamide | 2.83 (2H, d), 3.01 (1H, t), 3.07 (2H, s), | |
| | | | | 3.34 (2H, d), 3.64 (1H, t), | |
| | | | | 3.86-3.91 (1H, m), 6.53 (1H, d), 6.68 (2H, d), | |
| | | | | 6.98-7.03 (3H, m), 7.16-7.22 (1H, | |
| | | | | m), 7.78-7.80 (1H, m), | |
| | | | | 8.36-8.38 (1H, m) | |
| 27 | 4 | 2 | N-cyclohexyl-6- | 1.16-1.21 (2H, m), 1.22 (1H, s), | 355 |
| | | | methyl-2- | 1.30-1.37 (2H, m), 1.53-1.56 (1H, m), | |
| | | | phenethylsulfanyl- | 1.63-1.69 (2H, m), 1.91-1.95 (2H, m), | |
| | | | pyridine-3- | 2.50 (3H, s), 2.86-2.94 (2H, m), | |
| | | | carboxamide | 3.42-3.44 (1H, m), 3.44 (1H, d), | |
| | | | | 3.88-3.95 (1H, m), 6.38 (1H, d), 6.85 (1H, d), | |
| | | | | 7.11-7.15 (1H, m), 7.18-7.22 (4H, | |
| | | | | m), 7.72 (1H, d) | |
| 28 | 3 | 1 | N-cyclohexyl-2-[2- | 1.15-1.18 (1H, m), 1.22 (1H, s), | 419 |
| | | | (4- | 1.30-1.38 (2H, m), 1.58 (1H, d), 1.67 (1H, | |
| | | | methylsulfonylphenyl)ethylsulfanyl]pyridine- | d), 1.67-1.71 (1H, m), 1.93-1.97 (2H, | |
| | | | 3- | m), 2.96 (3H, s), 3.04 (2H, d), | |
| | | | carboxamide | 3.45 (2H, d), 3.88-3.95 (1H, m), 6.30 (1H, | |
| | | | | d), 7.04-7.07 (2H, m), 7.38 (2H, d), | |
| | | | | 7.74-7.76 (2H, m), 7.78 (1H, s), | |
| | | | | 8.44-8.45 (1H, m) | |
| 29 | 10 | — | [2-(2- | 1.48 (2H, d), 1.58-1.63 (2H, m), | 371 |
| | | | hydroxyethyl)-1- | 1.77 (1H, s), 1.96-2.00 (1H, m), 2.93 (3H, | |
| | | | piperidyl]-(2- | t), 3.15 (1H, d), 3.35-3.47 (3H, m), | |
| | | | phenethylsulfanylpyridin- | 3.60-3.63 (1H, m), 4.92 (1H, t), | |
| | | | 3- | 6.96-6.99 (1H, m), 7.10-7.15 (1H, m), | |
| | | | yl)methanone | 7.17-7.20 (1H, m), 7.18-7.23 (3H, m), | |
| | | | | 7.32 (1H, d), 8.40-8.41 (1H, m) | |
| 30 | 10 | — | (2- | 1.16 (1H, t), 1.54-1.92 (2H, m), | 359 |
| | | | phenethylsulfanylpyridin- | 2.27 (1H, s), 2.46 (2H, s), 2.84-2.90 (2H, | |
| | | | 3-yl)-(1,4- | m), 2.82-3.01 (2H, m), | |
| | | | thiazepan-4- | 3.11-3.21 (1H, m), 3.19-3.24 (1H, m), | |
| | | | yl)methanone | 3.30-3.35 (2H, m), 3.38 (1H, d), 3.44 (1H, | |
| | | | | d), 3.53-3.57 (1H, m), 3.88-3.93 (1H, | |
| | | | | m), 6.75 (1H, s), 6.84-6.89 (1H, m), | |
| | | | | 7.00-7.02 (1H, m), 7.09-7.21 (10H, | |
| | | | | m), 8.31-8.36 (1H, m) | |
| 31 | 11 | 1&5 | 2-[2-(2- | ¹H NMR (300.073 MHz, DMSO-d₆) | 366 |
| | | | cyanophenyl)ethylsulfanyl]- | δ1.06-1.18 (1H, m), 1.22-1.36 (4H, | |
| | | | N- | m), 1.58 (1H, m), 1.65-1.74 (2H, m), | |
| | | | cyclohexyl- | 1.81 (2H, m), 3.13 (2H, t), 3.38 (2H, t), | |
| | | | pyridine-3- | 3.64-3.73 (1H, m), 7.15-7.19 (1H, | |
| | | | carboxamide | m), 7.39-7.44 (1H, m), | |
| | | | | 7.54-7.56 (1H, m), 7.62-7.72 (2H, m), | |
| | | | | 7.76-7.79 (1H, m), 8.24 (1H, d), | |
| | | | | 8.49-8.51 (1H, m)- | |
| 32 | 11 | 1&6 | 2-[2-(3- | ¹H NMR (400.13 MHz, DMSO-d₆) | 366 |
| | | | cyanophenyl)ethylsulfanyl]- | δ1.10-1.19 (1H, m), 1.21-1.36 (4H, | |
| | | | N- | m), 1.57-1.60 (1H, m), | |
| | | | cyclohexyl- | 1.68-1.74 (2H, m), 1.82 (2H, m), 2.98 (2H, t), | |
| | | | pyridine-3- | 3.38 (2H, t), 3.66-3.73 (1H, m), | |
| | | | carboxamide | 7.17-7.20 (1H, m), 7.52 (1H, t), 7.64 (1H, d), | |
| | | | | 7.68-7.71 (2H, m), 7.75 (1H, s), | |
| | | | | 8.25 (1H, d), 8.52-8.54 (1H, m) | |
| 33 | 13 | 1 | N-cyclohexyl-2-[2- | ¹H NMR (300.073 MHz, DMSO-d₆) | 435 |
| | | | (3- | δ1.06-1.40 (5H, m), 1.56-1.60 (1H, | |
| | | | methylsulfonyloxy | m), 1.75-1.83 (4H, m), 2.96 (2H, t), | |
| | | | phenyl)ethylsulfanyl]pyridine- | 3.29-3.40 (5H, m), 3.66 (1H, m), | |
| | | | 3- | 7.15-7.21 (2H, m), 7.27-7.30 (2H, m), | |
| | | | carboxamide | 7.41 (1H, t), 7.67-7.70 (1H, m), | |
| | | | | 8.26 (1H, d), 8.51-8.53 (1H, m) | |
| 34 | 14 | Ex | N-cyclohexyl-2-[2- | ¹H NMR (300.072 MHz, CDCl₃) | 357 |
| | | 33 | (3- | δ1.15-1.49 (5H, m), 1.60-1.77 (3H, m), | |
| | | | hydroxyphenyl)ethylsulfanyl]pyridine- | 2.02 (2H, m), 2.89-2.94 (2H, t), | |
| | | | 3-carboxamide | 3.42-3.47 (2H, t), 3.94-4.06 (1H, m), | |
| | | | | 6.06 (1H, s), 6.25 (1H, d), 6.65-6.69 (1H, | |
| | | | | m), 6.74-6.77 (2H, m), | |
| | | | | 7.00-7.04 (1H, m), 7.12 (1H, t), 7.76-7.80 (1H, | |
| | | | | dd), 8.43-8.45 (1H, dd) | |
| 35 | 15 | Ex | 2-[2-(2- | ¹H NMR (400.13 MHz, DMSO-d₆) | 384 |
| | | 33 | carbamoylphenyl)ethylsulfanyl]- | δ1.10-1.19 (1H, m), 1.20-1.37 (4H, | |
| | | | N- | m), 1.59 (1H, m), 1.71-1.74 (2H, m), | |
| | | | cyclohexyl- | 1.83 (2H, m), 3.04 (2H, m), | |
| | | | pyridine-3- | 3.32-3.36 (2H, m), 3.67-3.74 (1H, m), | |
| | | | carboxamide | 7.15-7.18 (1H, m), 7.25-7.31 (1H, m), | |
| | | | | 7.34-7.40 (4H, m), 7.67-7.69 (1H, m), | |
| | | | | 7.74 (1H, s), 8.26 (1H, d), | |
| | | | | 8.52-8.53 (1H, m) | |

*In this case purification was by column chromatography, eluting with 40 to 90% EtOAc in IH

1. A compound of formula (1): 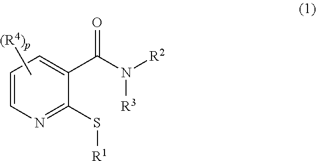
wherein:
R¹ is selected from phenylC₂₋₄alkyl, heteroarylC₂₋₄alkyl, phenylC₃₋₇cycloalkyl and heteroarylC₃₋₇cycloalkyl [each of which is optionally substituted on the ring, alkyl or cycloalkyl group by 1, 2 or 3 substitutents independently selected from C₁₋₃alkyl, C₂₋₃alkenyl, C₂₋₃alkynyl, hydroxy, halo, oxo, cyano, trifluoromethyl, C₁₋₃alkoxy, C₁₋₃alkylS(O)ₙ— (wherein n is 0, 1, 2 or 3), C₁₋₃alkylS(O)ₜO— (wherein t is 0, 1, 2 or 3), R⁵CON(R^{5′})—, (R^{5′})(R^{5″})NC(O)—, R^{5′}OC(O)— and (R^{5′})(R^{5″})NSO₂— (wherein R⁵ is C₁₋₃alkyl optionally substituted by hydroxyl, halo or cyano)]; and
R^{5′} and R^{5″} are independently selected from hydrogen and C₁₋₃alkyl optionally substituted by hydroxyl, halo, C₁₋₃alkoxy, carboxy or cyano)];
R² is selected from C₃₋₇cycloalkyl(CH₂)ₘ—, C₆₋₁₂bicycloalkyl(CH₂)ₘ— and C₆₋₁₂tricycloalkyl(CH₂)ₘ— (wherein m is 0, 1 or 2 and the cycloalkyl, bicycloalkyl and tricycloalkyl rings are optionally substituted by 1, 2 or 3 substituents independently selected from R⁶);
R³ is selected from hydrogen and C₁₋₄alkyl;
R² and R³ together with the nitrogen atom to which they are attached form a saturated mono, bicyclic or bridged ring system optionally containing 1 or 2 additional ring heteroatoms selected from nitrogen, oxygen and sulphur and which is optionally fused to a saturated, partially saturated or unsaturated monocyclic ring wherein the resulting ring system is optionally substituted by 1, 2, or 3 substituents independently selected from R⁷;
R⁴ is selected from C₁₋₄alkyl, C₂₋₄alkenyl, C₂₋₄alkynyl, hydroxyl, cyano, C₁₋₄alkanoyl, trifluoromethyl, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)_{q}—, C₁₋₄alkylS(O)_{q}C₁₋₄alkyl (wherein q is 0, 1 and 2) or a 4-7 membered saturated heterocyclic ring having 1 mandatory ring nitrogen and optionally an additional ring heteroatom selected from nitrogen, oxygen and sulphur (wherein any ring or alkyl group in each of the aforementioned groups is optionally substituted by 1, 2 or 3 substituents independently selected from R⁸);
R⁶, R⁷ and R⁸ are independently selected from hydroxyl, halo, oxo, carboxy, cyano, trifluoromethyl, R⁹, R⁹O—, R⁹CO—, R⁹C(O)O—, R⁹CON(R^{9′})—, (R^{9′})(R^{9″})NC(O)—, (R^{9′})(R^{9″})N—, R⁹S(O)ₐ— wherein a is 0 to 2, R^{9′}OC(O)—, (R^{9′})(R^{9″})NSO₂—, R⁹SO₂N(R^{9″})—, (R^{9′})(R^{9″})NC(O)N(R^{9′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted by 1, 2 or 3 substituents independently selected from C₁₋₄alkyl, hydroxyl, cyano, trifluoromethyl, trifluoromoxy, halo, C₁₋₄alkoxy, C₁₋₄alkoxyC₁₋₄alkyl, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ—, C₁₋₄alkylS(O)ᵣC₁₋₄alkyl (wherein r is 0, 1 and 2)];
R⁹ is C₁₋₃alkyl optionally substituted by hydroxyl, halo, C₁₋₄alkoxy, carboxy or cyano;
R^{9′}, R^{9″} and R^{9′″} are independently selected from hydrogen and C₁₋₃alkyl optionally substituted by hydroxyl, halo, C₁₋₄alkoxy, carboxy or cyano);
p is 0, 1 or 2;
or an in vivo hydrolysable ester or a pharmaceutically-acceptable salt thereof;
provided that the compound of the formula (1) is not:
1-{2-[(3,5-dimethyl-4-isoxazolyl)methylthio]-3-pyridylcarbonyl}-2-(2-thienyl)pyrrolidine;
N-cyclohexyl-2-(phenethylsulfanyl)-6-trifluoromethylpyridine-3-carboxamide; or
N-cyclohexyl-2-[2-(2-carboxyphenyl)ethyl)sulfanyl]pyridine-3-carboxamide.

2. A compound according to claim 1 wherein R¹ is phenylethyl or 2-(pyridyl)ethyl [each being optionally substituted by 1, 2 or 3 substituents independently selected from C₁₋₃alkyl, C₂₋₃alkenyl, C₂₋₃alkynyl, hydroxy, halo, oxo, cyano, trifluoromethyl, C₁₋₃alkoxy, C₁₋₃alkylS(O)ₙ— (wherein n is 0, 1, 2 or 3), C₁₋₃alkylS(O)ₜO— (wherein t is 0, 1, 2 or 3), R⁵CON(R^{5′})—, (R^{5′})(R^{5″})NC(O)—, R^{5′}OC(O)— and (R^{5′})(R^{5″})NSO₂— (wherein R⁵ is C₁₋₃alkyl optionally substituted by hydroxyl, halo or cyano; and
R^{5′} and R^{5″} are independently selected from hydrogen and C₁₋₃alkyl optionally substituted by hydroxyl, halo, C₁₋₄alkoxy, carboxy or cyano)].

3. A compound according to claim 1 wherein R³ is hydrogen or methyl.

4. A compound according to claim 1 of formula (1′): 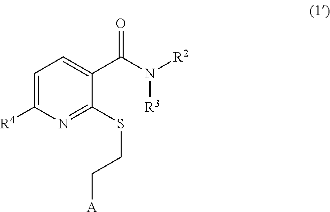
wherein:
A is selected from phenyl or heteroaryl [each of which is optionally substituted by 1, 2 or 3 substitutents independently selected from C₁₋₃alkyl, C₂₋₃alkenyl, C₂₋₃alkynyl, hydroxy, halo, oxo, cyano, trifluoromethyl, C₁₋₃alkoxy, C₁₋₃alkylS(O)ₙ— (wherein n is 0, 1, 2 or 3), C₁₋₃alkylS(O)ₜO— (wherein t is 0, 1, 2 or 3), R⁵CON(R^{5′})—, (R^{5′})(R^{5″})NC(O)—, R^{5′}OC(O)— and (R^{5′})(R^{5″})NSO₂— (wherein R⁵ is C₁₋₃alkyl optionally substituted by hydroxyl, halo or cyano)]; and
R^{5′} and R^{5″} are independently selected from hydrogen and C₁₋₃alkyl optionally substituted by hydroxyl, halo, C₁₋₃alkoxy, carboxy or cyano)];
R² is selected from C₃₋₇cycloalkyl(CH₂)ₘ—, C₆₋₁₂bicycloalkyl(CH₂)ₘ— and C₆₋₁₂tricycloalkyl(CH₂)ₘ— (wherein m is 0, 1 or 2 and the cycloalkyl, bicycloalkyl and tricycloalkyl rings are optionally substituted by 1, 2 or 3 substituents independently selected from R⁶);
R³ is selected from hydrogen and methyl; or
R² and R³ together with the nitrogen atom to which they are attached form a saturated mono, bicyclic or bridged ring system optionally containing 1 or 2 additional ring heteroatoms selected from nitrogen, oxygen and sulphur and which is optionally fused to a saturated, partially saturated or unsaturated monocyclic ring wherein the resulting ring system is optionally substituted by 1, 2, or 3 substituents independently selected from R⁷;
R⁴ is selected from C₁₋₄alkyl, C₂₋₄alkenyl, C₂₋₄alkynyl, hydroxyl, cyano, C₁₋₄alkanoyl, trifluoromethyl, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)_{q}—, C₁₋₄alkylS(O)_{q}C₁₋₄alkyl (wherein q is 0, 1 and 2) or a 4-7 membered saturated heterocyclic ring having 1 mandatory ring nitrogen and optionally an additional ring heteroatom selected from nitrogen, oxygen and sulphur (wherein any ring or alkyl group in each of the aforementioned groups is optionally substituted by 1, 2 or 3 substituents independently selected from R⁸);
R⁶, R⁷ and R⁸ are independently selected from hydroxyl, halo, oxo, carboxy, cyano, trifluoromethyl, R⁹, R⁹O—, R⁹CO—, R⁹C(O)O—, R⁹CON(R^{9′})—, (R^{9′})(R^{9″})NC(O)—, (R^{9′})(R^{9″})N—, R⁹S(O)ₐ— wherein a is 0 to 2, R^{9′}OC(O)—, (R^{9′})(R^{9″})NSO₂—, R⁹SO₂N(R^{9″})—, (R^{9′})(R^{9″})NC(O)N(R^{9′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted by 1, 2 or 3 substituents independently selected from C₁₋₄alkyl, hydroxyl, cyano, trifluoromethyl, trifluoromoxy, halo, C₁₋₄alkoxy, C₁₋₄alkoxyC₁₋₄alkyl, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ—, C₁₋₄alkylS(O)ᵣC₁₋₄alkyl (wherein r is 0, 1 and 2)];
R⁹ is C₁₋₃alkyl optionally substituted by hydroxyl, halo, C₁₋₄alkoxy, carboxy or cyano;
R^{9′}, R^{9″} and R^{9′″} are independently selected from hydrogen and C₁₋₃alkyl optionally substituted by hydroxyl, halo, C₁₋₄alkoxy, carboxy or cyano);
or an in vivo hydrolysable ester or a pharmaceutically-acceptable salt thereof:
provided that the compound of the formula (1) is not:
N-cyclohexyl-2-(phenethylsulfanyl)-6-trifluoromethylpyridine-3-carboxamide; or
N-cyclohexyl-2-[2-(2-carboxyphenyl)ethyl)sulfanyl]pyridine-3-carboxamide.

5. A compound according to claim 1 wherein R² is selected from C₅₋₇cycloalkyl(CH₂)ₘ—, C₇₋₁₀bicycloalkyl(CH₂)ₘ— and adamantyl (wherein the cycloalkyl, bicycloalkyl and adamantyl rings are optionally substituted by 1, 2 or 3 substituents independently selected from R⁶ wherein R⁶ is as defined in claim 1) and wherein m is 0, 1 or 2.

6. A compound according to claim 1 wherein R² and R³ together with the nitrogen atom to which they are attached form a saturated 5 or 6-membered mono, 6-12 membered bicyclic or 6-12 membered bridged ring system optionally containing 1 or 2 additional ring heteroatoms selected from nitrogen, oxygen and sulphur and which is optionally fused to a saturated, partially-saturated or aryl monocyclic ring wherein the resulting ring system is optionally substituted by 1, 2, or 3 substituents independently selected from R⁷ wherein R⁷ is as defined in claim 1.

7. A compound according to claim 1 wherein R⁴ is selected from C₁₋₄alkyl, hydroxyl, cyano, C₁₋₄alkanoyl, trifluoromethyl, halo, C₁₋₄alkoxy, C₁₋₄alkoxyC₁₋₄alkyl, N—C₁₋₄alkylamino, di-N,N—(C₁₋₄alkyl)amino or a 4-7 membered saturated heterocyclic ring having 1 mandatory ring nitrogen and optionally an additional ring heteroatom selected from nitrogen, oxygen and sulphur (wherein any ring or alkyl group in each of the aforementioned groups is optionally substituted by 1, 2 or 3 substituents independently selected from R⁸ and R⁸ is as defined in claim 1).

8. A compound as defined in any one of claims 1 to 3 and 5 to 7 wherein p is 1 and R⁴ is a substituent in the 6-position of the pyridine ring.

9. A compound according to claim 1, selected from:
N-cyclohexyl-2-(3-phenylpropylsulfanyl)pyridine-3-carboxamide;
N-cyclohexyl-2-(2-furylmethylsulfanyl)pyridine-3-carboxamide;
N-cyclohexyl-2-(2-pyridin-2-ylethylsulfanyl)pyridine-3-carboxamide;
N-cyclohexyl-2-phenethylsulfanyl-pyridine-3-carboxamide;
N-cyclohexyl-2-(2-phenylpropylsulfanyl)pyridine-3-carboxamide N-Cyclohexyl-5-fluoro-2-phenethylsulfanyl-pyridine-3-carboxamide;
N-cyclohexyl-5-fluoro-2-phenethylsulfanyl-pyridine-3-carboxamide;
N-cyclohexyl-2-phenacylsulfanyl-pyridine-3-carboxamide;
N-cyclohexyl-2-(2-hydroxy-2-phenyl-ethyl)sulfanyl-pyridine-3-carboxamide;
N-cyclohexyl-2-[2-(4-methylsulfonyloxyphenyl)ethylsulfanyl]pyridine-3-carboxamide;
[3-(2-hydroxyethyl)-1-piperidyl]-(2-phenethylsulfanylpyridin-3-yl)methanone;
N-cyclohexyl-2-(2-pyridin-3-ylethylsulfanyl)pyridine-3-carboxamide;
6-chloro-N-cyclohexyl-2-phenethylsulfanyl-pyridine-3-carboxamide;
N-cyclohexyl-2-[2-(2-methylsulfonyloxyphenyl)ethylsulfanyl]pyridine-3-carboxamide;
N-cyclohexyl-2-[2-(2-hydroxyphenyl)ethylsulfanyl]pyridine-3-carboxamide;
2-[2-(3-carbamoylphenyl)ethylsulfanyl]-N-cyclohexyl-pyridine-3-carboxamide;
3-[2-[3-(cyclohexylcarbamoyl)pyridin-2-yl]sulfanylethyl]benzoic acid;
cis-4-[(6-methyl-2-phenethylsulfanyl-pyridine-3-carbonyl)amino]cyclohexane-1-carboxylic acid;
4-[methyl-(6-methyl-2-phenethylsulfanyl-pyridine-3-carbonyl)amino]cyclohexane-1-carboxylic acid;
6-chloro-N-[(2r,5s)-5-hydroxy-2-adamantyl]-2-phenethylsulfanyl-pyridine-3-carboxamide; and
(1r,4s)-4-[({6-methyl-2-[(2-phenylethyl)thio]pyridin-3-yl}carbonyl)amino]adamantane-1-carboxylic acid;
or a pharmaceutically-acceptable salt thereof.

10. A pharmaceutical composition, which comprises a compound of formula (I), or a pharmaceutically-acceptable salt thereof, as claimed in claim 1 in association with a pharmaceutically-acceptable diluent or carrier.

11-13. (canceled)

14. A process for preparing a compound of the formula (1) or a pharmaceutically acceptable salt or in vivo hydrolysable ester thereof as claimed in claim 1, which process [wherein variable groups are, unless otherwise specified, as defined in claim 1] comprises any one of processes a) to c):
a) reaction of a compound of formula (2) with a compound of formula (3): 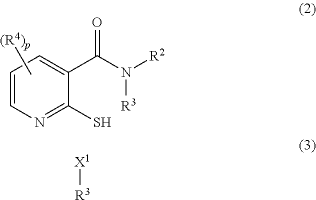
wherein X¹ is a leaving group; or
b) reaction of a compound of formula (4) with a compound of formula (5): 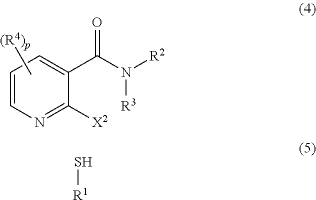
wherein X² is a leaving group; or
c) reaction of a compound of formula (6) with a compound of formula (7): 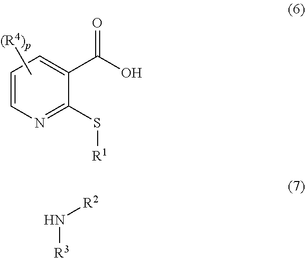
and thereafter optionally:
i) converting a compound of the formula (1) into another compound of the formula (1);
ii) removing any protecting groups;
iii) resolving enantiomers;
iv) forming a salt or in vivo hydroysable ester thereof.

15. A method of producing an 11βHSD1 inhibitory effect, in a warm-blooded animal in need of such treatment, which comprises administering to said animal an effective amount of a compound of formula (1) or a pharmaceutically acceptable salt thereof, as claimed in claim 1.

16. A method of treating or preventing a disease associated with 11βHSD1 activity, comprising administering an effective amount of a compound of formula (I) according to claim 1 or a pharmaceutically-acceptable salt, to a warm-blooded animal need of such treatment.

17. The method of claim 15 or 16, wherein the animal is a human.
